(19) United States
(12) Patent Application Publication
(10) Pub. No.: US 2006/0258644 A1
(43) Pub. Date: Nov. 16, 2006

(54) Pyrrolobenzodiazepines and heteroaryl, aryl and cycloalkylamino ketone derivatives as follicle stimulating hormone receptor (FSH-R) antagonists

(75) Inventors: Amedeo A. Failli, Princeton Junction, NJ (US); Arthur A. Santilli, Havertown, PA (US); Dominick A. Quagliato, Bridgewater, NJ (US); Emily S. Shen, West Chester, PA (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/432,177

(22) Filed: May 11, 2006

Related U.S. Application Data
(60) Provisional application No. 60/680,321, filed on May 12, 2005.

(51) Int. Cl. A61K 31/551; C07D 487/04

(52) U.S. Cl. 514/220; 540/558

(57) ABSTRACT

The invention provides compounds of formula 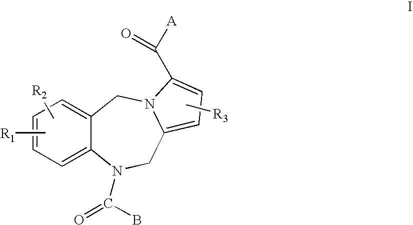
or a pharmaceutically acceptable salt thereof, wherein R, R₁, R₂, R₃, A, and B are as defined in the accompanying specification. Methods of making such compounds are also provided.

This application claims benefit of priority to U.S. Provisional Patent Application No. 60/680,321 filed May 12, 2005, which is hereby incorporated by reference.

### FIELD OF THE INVENTION

The present invention relates to pyrrolobenzodiazepines and derivatives thereof having antagonist activity on the FSH receptor, and to their use as contraceptives.

### BACKGROUND OF THE INVENTION

Reproduction in women depends upon the dynamic interaction of several compartments of the female reproductive system. The hypothalamic-pituitary-gonadal axis orchestrates a series of events affecting the ovaries and the uterine-endometrial compartment that leads to the production of mature ova, ovulation, and ultimately appropriate conditions necessary for fertilization. Specifically, luteinizing hormone-releasing hormone (LHRH), released from the hypothalamus, initiates the release of the gonadotropins, luteneizing hormone (LH) and follicle stimulating hormone (FSH) from the pituitary. These hormones act directly on the ovary to promote the development of selected follicles by inducing granulosa and theca cell proliferation and differentiation. FSH stimulates aromatization of androgens to estrogens and increases the expression of LH receptors in the theca cells. The follicles, in turn, secrete steroids (estradiol, progesterone) and peptides (inhibin, activin). Estradiol and inhibin levels progressively increase during the follicular phase of the menstrual cycle until ovulation. Inhibin decreases FSH secretion from the pituitary gland, while estradiol acts on the hypothalamus and pituitary to induce the LH surge in mid-cycle, which results in ovulation. Afterwards, the post-ovulation, ruptured follicle forms the corpus luteum, which produces progesterone. Ovarian hormones, in turn, regulate the secretion of gonadotropins through a classical long-loop negative feedback mechanism. The elucidation of these control mechanisms has provided opportunities for the development of effective strategies to control fertility, including both enhancement of fertility and contraception. For recent reviews of FSH action see: “FSH Action and Intraovarian Regulation”, B. C. J. M. Fauser Editor, Parthenon Publishing Group, Vol. 6, 1997 and A. J. Hsueh, T. Bicsak, X.-C. Ja, K. D. Dahl, B. C. J. M. Fauser, A. B. Galway, N. Czwkala, S. Pavlou, H. Pakoff, J. Keene, I. Boime, Granulosa “Cells as Hormone Targets: The Role of Biologically Active Follicle-Stimulating Hormone in Reproduction”, Rec. Prog. Horm. Res., 45, 209-227,1989.

Current hormonal contraception methods are steroidal in nature (progestins and estrogens) and modulate long-loop feedback inhibition of gonadotropin secretion, as well as affecting peripheral mechanisms such as sperm migration and fertilization. The development of specific antagonists of the receptor for FSH (FSH-R) would provide an alternative strategy for hormonal contraception. Such antagonists would block FSH-mediated follicular development leading to a blockade of ovulation, thereby producing the desired contraceptive effect. Support for the effectiveness of this strategy is provided by the mechanism that causes resistant ovary syndrome which results in infertility in women. The infertility experienced by these women is the result of non-functional FSH receptors (K. Aittomaki, J. L. D. Lucena, P. Pakarinen, P. Sistonen, J. Tapainainnen, J. Gromoll, R. Kashikari, E.-M. Sankila, H. Lehvaslaiho, A. R. Engel, E. Nieschlag, I. Huhtaniemi, A. de la Chapelle “Mutations in the Follicle-Stimulating Hormone Receptor Gene Causes Hereditary Hypergonadotropic Ovarian Failure” Cell, 82, 959-968, 1995). This approach to contraception may be applicable to men as well, since idiopathic male infertility seems to be related to a reduction in FSH binding sites. In addition, men with selective FSH deficiency are oligo- or azoospermic with normal testosterone levels and present normal virilization (G. Lindstedt, E. Nystrom, C. Matthews, l. Ernest, P. O. Janson, K. Chattarjee, Clin. Lab. Med., 36, 664, 1998). Therefore, orally active, low molecular weight FSH antagonists may provide a versatile novel method of contraception. Such an antagonist could be expected to interfere with follicle development and thus ovulation, while maintaining sufficient estrogen production and beneficial effects on bone mass.

FSH actions are mediated by binding of the hormone to a specific transmembrane G protein-coupled receptor exclusively expressed in the ovary, leading to activation of the adenyl cyclase system and elevation of intracellular levels of the second messenger cAMP (A. Mukherjee, O. K. Park-Sarge, K. Mayo, Endocrinology, 137, 3234 (1996)).

### SUMMARY OF THE INVENTION

In some embodiments, the invention provides compounds represented by the formula I 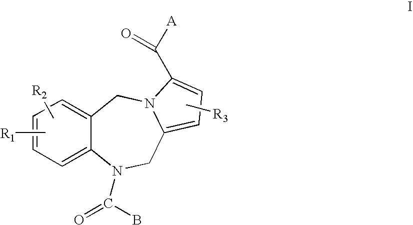
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];

R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B is B₁ or B₂,

wherein B₁ is selected independently from the group consisting of

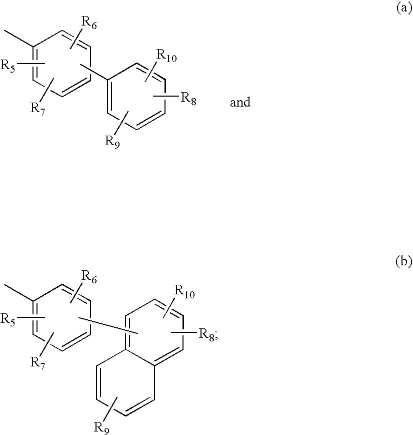

wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxyalkyl, hydroxy(C₁-C₆) alkyl, alkyloxyalkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈)cycloalkyloxycarbonyl, aryl(C₁-C₆)alkyloxycarbonyl, carbamoyl, —O—CH₂—CH═CH₂, (C₁-C₆)alkyl substituted with 1-3 halogen atoms, trihalomethyl, trifluoromethyl, halogen, OCF₃, thioalkyl, thio(C₁-C₆) alkyl, —C(O) alkyl, —C(O)aryl optionally substituted by alkyl; hydroxy, —CH(OH)alkyl, —CH(alkoxy)alkyl, nitro, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCONR₁₃R₁₄, —(CH₂)ₚCOOR₁₂, —CH═NOH, —CH═NO—(C₁-C₆) alkyl, trifluoromethylthio,

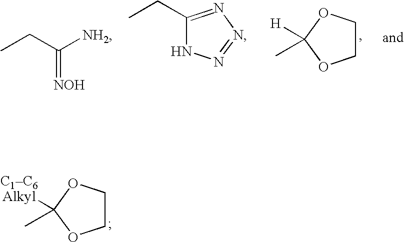

R₁₁ and R₁₂ are each independently hydrogen, alkyl, cycloalkyl, or C₃-C₈ cycloalkyl;

R₁₃ and R₁₄ are each independently hydrogen, alkyl, cycloalkyl, or C₃-C₈ cycloalkyl;

or R₁₃ and R₁₄ can be taken together with the nitrogen to which they are attached to form a 4-6 membered saturated ring optionally containing up to two atoms selected from O, S or N;

p is 0 or 1;
A is A₁ or A₂, wherein

A₁ is selected from

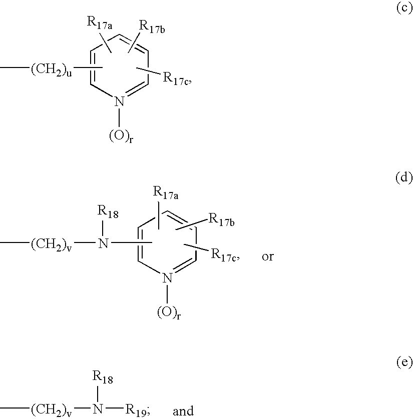

A₂ is selected from 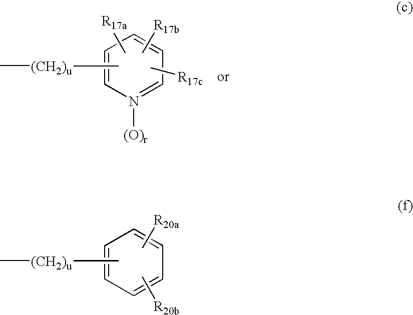
provided that when A is A₂, then B is B₂ wherein B₂ is

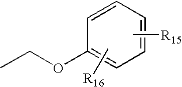

wherein R₁₅ and R₁₆ are selected independently from the group consisting of hydrogen, alkyl, and halogen;

wherein

R₁₇ₐ, R_{17b}, and R_{17c} are each independently selected from the group consisting of hydrogen, alkyl, halogen, hydroxy, aryloxy, and hydroxyalkyl;

u is the integer 0, 1, 2, 3, or 4;

v is the integer 1, 2, 3, or 4;

r is 0 or 1;

R₁₈ is hydrogen or alkyl; and

R₁₉ is a cycloalkylamine.

R₂₀ₐ and R_{20b} are each independently selected from the group consisting of hydrogen, alkyl, halogen, or aryl; or R₂₀ₐ and R_{20b} can be taken together with the aryl to which they are attached to form an aromatic bicycle having up to 10 total ring atoms.

In some embodiments, the invention provides compounds represented by the formula II 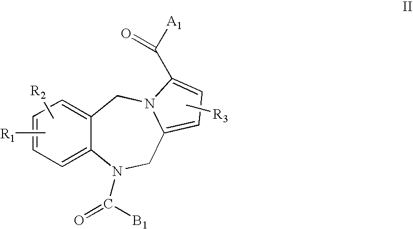
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, -OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];

R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B₁ is selected independently from the group consisting of 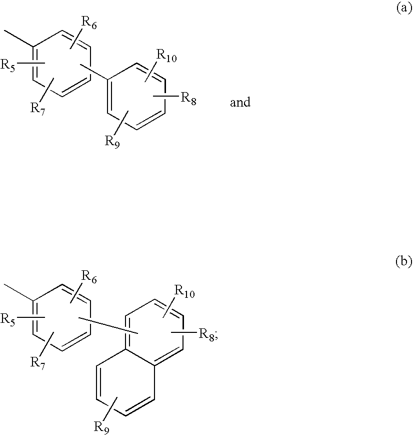
   wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxyalkyl, hydroxy(C₁-C₆) alkyl, alkyloxyalkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, (C₁-C₆)alkyl substituted with 1-3 halogen atoms, trihalomethyl, trifluoromethyl, halogen, OCF₃, thioalkyl, thio(C₁-C₆) alkyl, —C(O) alkyl, —C(O)aryl optionally substituted by alkyl; hydroxy, —CH(OH)alkyl, —CH(alkoxy)alkyl, nitro, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH [(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCONR₁₃R₁₄, —(CH₂)ₚCOOR₁₂, —CH═NOH, —CH═NO—(C₁-C₆) alkyl, trifluoromethylthio,

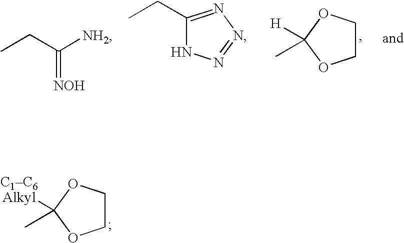

R₁₁ and R₁₂ are each independently hydrogen, alkyl, cycloalkyl, or C₃-C₈ cycloalkyl;

R₁₃ and R₁₄ are each independently hydrogen, alkyl, cycloalkyl, or C₃-C₈ cycloalkyl;

or R₁₃ and R₁₄ can be taken together with the nitrogen to which they are attached to form a 4-6 membered saturated ring optionally containing up to two atoms selected from O, S or N;

p is 0 or 1;
A₁ is selected from the group consisting of

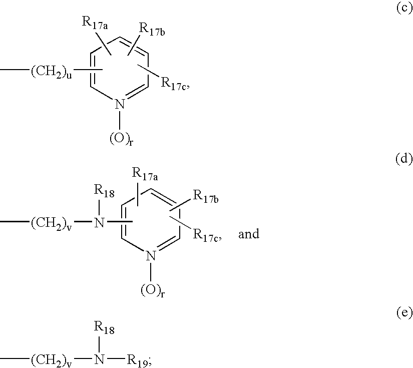

R₁₇ₐ, R_{17b}, and R_{17c} are each independently selected from the group consisting of hydrogen, alkyl, halogen, hydroxy, aryloxy, and hydroxyalkyl;

u is 0, 1, 2, 3, or 4;

v is 1, 2, 3, or 4;

r is 0 or 1;

R₁₈ is hydrogen or alkyl; and

R₁₉ is a cycloalkylamine.

In some embodiments, the invention provides compounds represented by the following formulae:

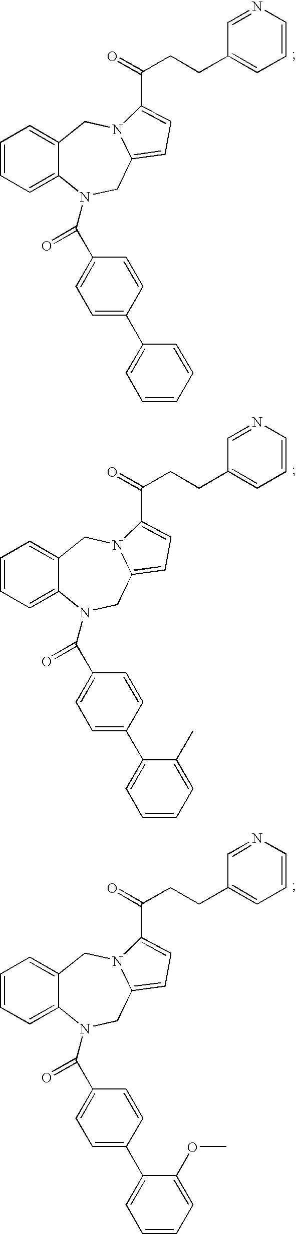

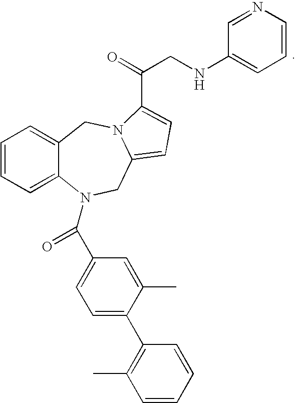

In some embodiments, the invention provides compounds represented by the following formula III: 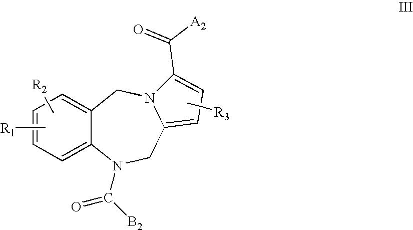
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];

R₃ is a substituent selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B₂ is

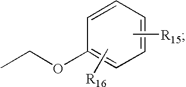

R₁₅ and R₁₆ are selected independently, from the group consisting of hydrogen, alkyl, and halogen;
and A₂ is selected from the group consisting of

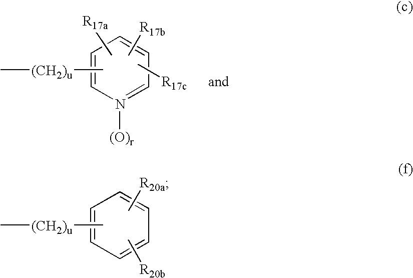

R₁₇ₐ, R_{17b}, and R_{17c} rare each independently selected from the group consisting of hydrogen, alkyl, halogen, hydroxy, aryloxy, and hydroxyalkyl;

u is 0, 1, 2, 3, or 4;

r is 0 or 1;

R₂₀ₐ and R_{20b} are independently selected from the group consisting of hydrogen, alkyl, halogen, and aryl; or

R₂₀ₐ and R_{20b} can be taken together with the aryl to which they are attached to form an aromatic bicycle having up to 10 ring atoms.

In some embodiments, the invention provides compounds represented by the following formulae:

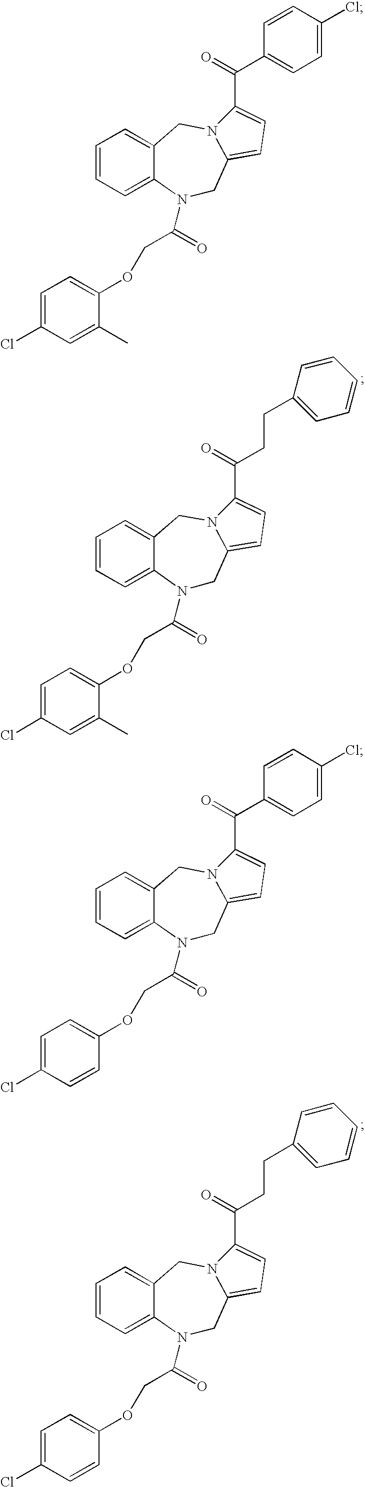

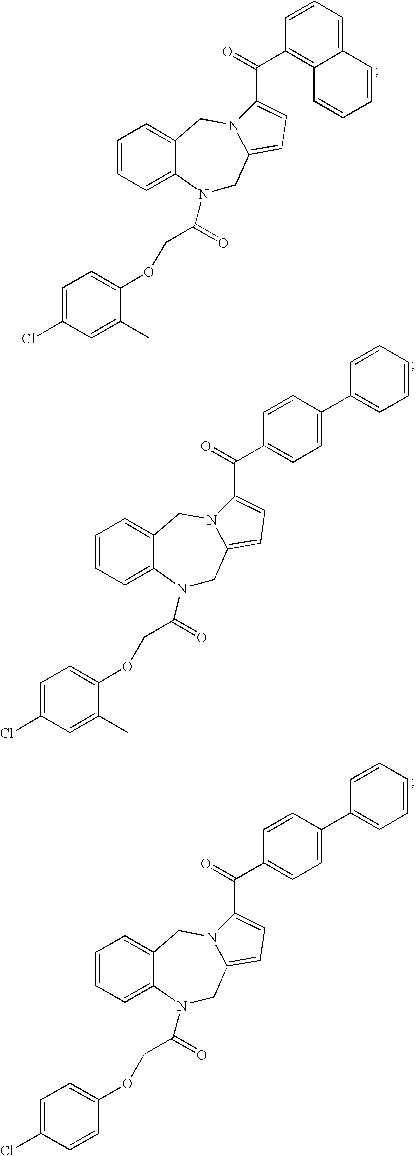

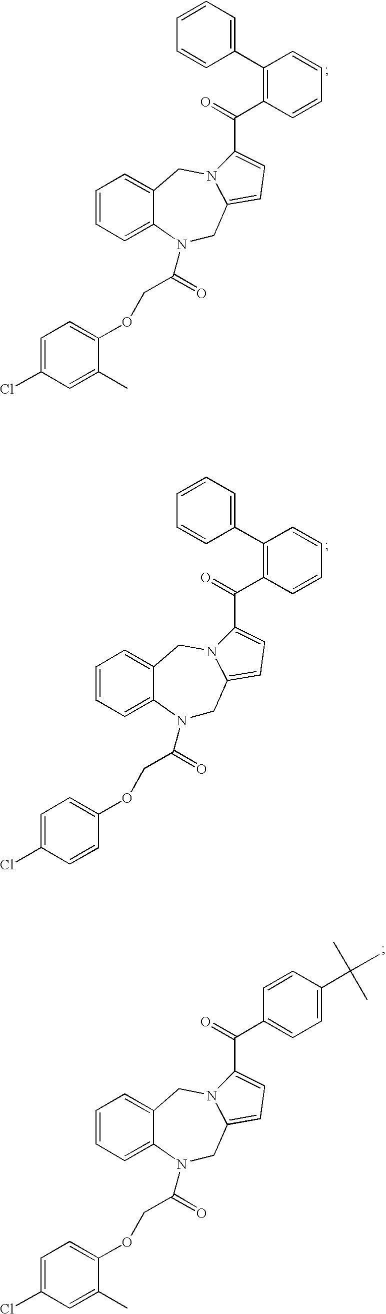

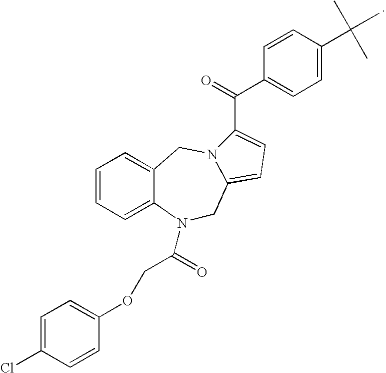

In some embodiments, the invention provides methods of preparing a compound of formula I 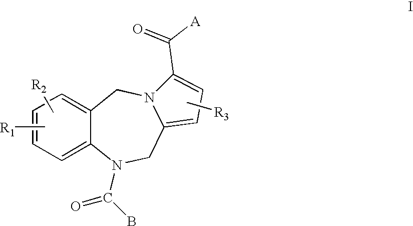
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];

R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B is B₁ or B₂,

wherein B₁ is selected independently from the group consisting of

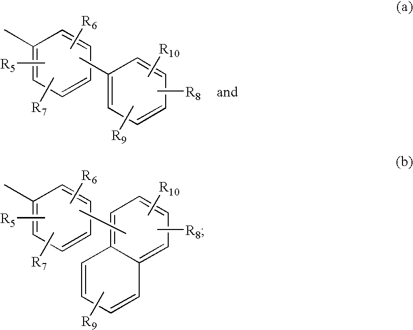

wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxy(C₁-C₆) alkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, halo (C₁-C₆)alkyl including trifluoromethyl, trihalomethyl, halogen, OCF₃, S((C₁-C₆) alkyl), —C(O) alkyl, —C(O)aryl optionally substituted by alkyl; hydroxy, hydroxyalkyl, alkyloxyalkyl, —CH(OH)alkyl, —CH(alkoxy)alkyl, formyl, nitro, thioalkyl, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCONR₁₃R₁₄, —(CH₂)ₚCOOR₁₂, —CH═NOH, —CH═NO—(C₁-C₆) alkyl, trifluoromethylthio, 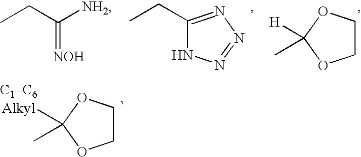
phenyl and naphthyl;

R₁₁ and R₁₂ are each independently hydrogen or alkyl;

R₁₃ and R₁₄ are each independently hydrogen or alkyl,

or R₁₃ and R₁₄ can be taken together with the nitrogen to which they are attached to form a 4-6 membered saturated ring optionally containing up to two atoms selected from O, S or N;

p is 0 or 1;
A is A₁ or A₂, wherein

A₁ is selected from

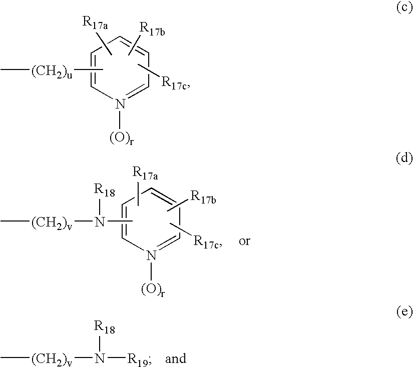

A₂ is selected from 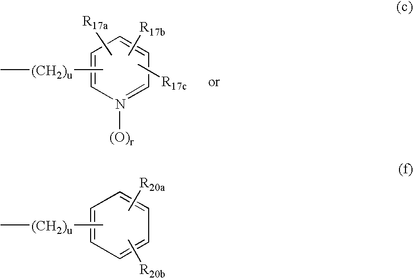
provided that when A is A₂, then B is B₂ wherein B₂ is

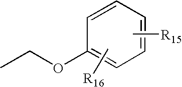

wherein R₁₅ and R₁₆ are selected independently from the group consisting of hydrogen, alkyl, and halogen;

wherein

R₁₇ₐ, R_{17b}, and R_{17c} are each independently selected from the group consisting of hydrogen, alkyl, halogen, hydroxy, aryloxy, and hydroxyalkyl;

u is the integer 0, 1, 2, 3, or 4;

v is the integer 1, 2, 3, or 4;

r is 0 or 1;

R₁₈ is hydrogen or alkyl; and

R₁₉ is a cycloalkylamine.

R₂₀ₐ and R_{20b} are each independently selected from the group consisting of hydrogen, alkyl, halogen, or aryl; or R₂₀ₐ and R_{20b} can be taken together with the aryl to which they are attached to form a bicyclic system; said method comprising:

reacting a tricyclic diazepine of formula (1) 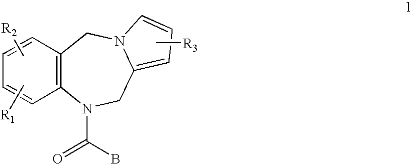
with an acyl halide of formula (4) 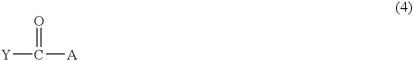
where Y is halogen;

under conditions sufficient to produce the desired compound of formula I.

In some embodiments, the invention provides methods for making a compound of formula (I) 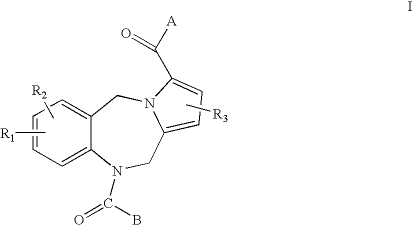
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];

R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B is B₁ or B₂,

wherein B₁ is selected independently from the group consisting of

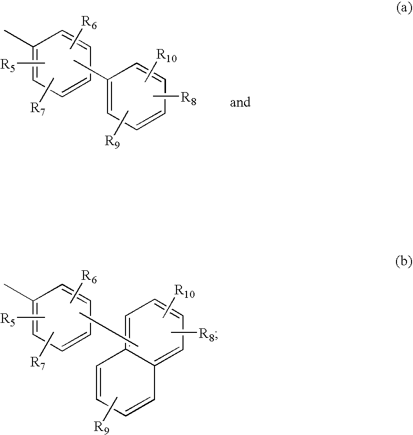

wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxy(C₁-C₆) alkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, halo (C₁-C₆)alkyl including trifluoromethyl, trihalomethyl, halogen, OCF₃, S((C₁-C₆) alkyl), —C(O) alkyl, —C(O)aryl optionally substituted by alkyl; hydroxy, hydroxyalkyl, alkyloxyalkyl, —CH(OH)alkyl, —CH(alkoxy)alkyl, formyl, nitro, thioalkyl, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCON R₁₃R₁₄, —(CH₂)ₚCOOR₁₂, —CH═NOH, —CH═NO—(C₁-C₆) alkyl, trifluoromethylthio, 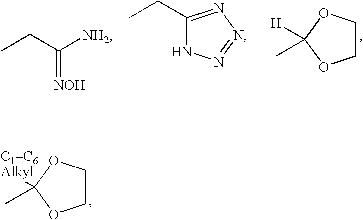
phenyl and naphthyl;

R₁₁ and R₁₂ are each independently hydrogen or alkyl;

R₁₃ and R₁₄ are each independently hydrogen or alkyl,

or R₁₃ and R₁₄ can be taken together with the nitrogen to which they are attached to form a 4-6 membered saturated ring optionally containing up to two atoms selected from O, S or N;

p is 0 or 1;
A is A₁ or A₂, wherein

A₁ is selected from

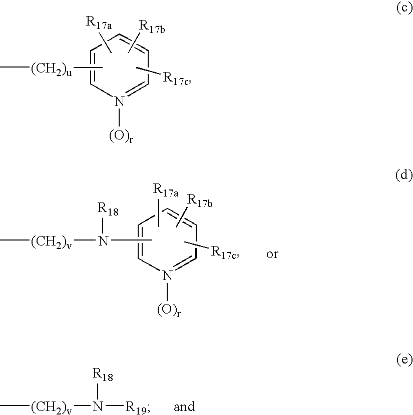

A₂ is selected from 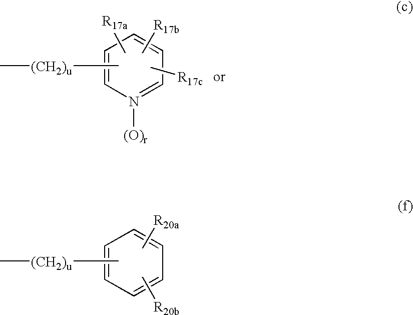
provided that when A is A₂, then B is B₂ wherein B₂ is

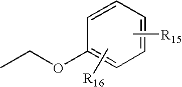

wherein R₁₅ and R₁₆ are selected independently from the group consisting of hydrogen, alkyl, and halogen;

wherein

R₁₇ₐ, R_{17b}, and R_{17c} are each independently selected from the group consisting of hydrogen, alkyl, halogen, hydroxy, aryloxy, and hydroxyalkyl;

u is the integer 0, 1, 2, 3, or 4;

v is the integer 1, 2, 3, or 4;

r is 0 or 1;

R₁₈ is hydrogen or alkyl; and

R₁₉ is a cycloalkylamine.

R₂₀ₐ and R_{20b} are each independently selected from the group consisting of hydrogen, alkyl, halogen, or aryl; or R₂₀ₐ and R_{20b} can be taken together with the aryl to which they are attached to form an aromatic bicycle having up to 10 total ring atoms;

said method comprising

subsequent reaction of the intermediate of formula (26) 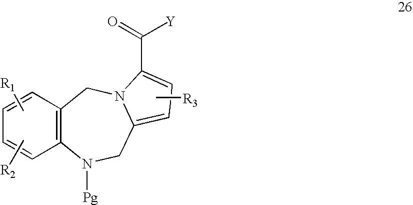
where Y is Cl, with an appropriate amine selected from 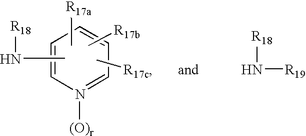
under the conditions sufficient to provide the intermediate of formula (27)

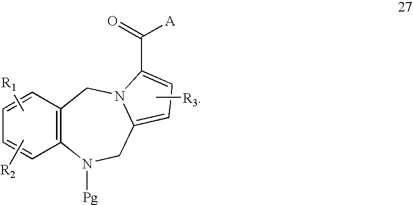

In some embodiments, the invention provides such methods further comprising deprotecting the compound of formula (27) to yield the intermediate of formula (28) 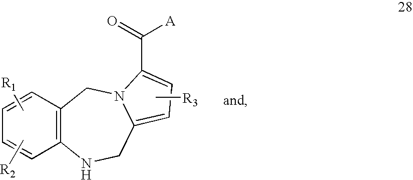
then acylating the intermediate of formula (28) to the desired product of formula (I).

In some embodiments, the invention provides methods wherein the compound of formula (26) is prepared by reacting a tricyclic diazepine of formula (25) 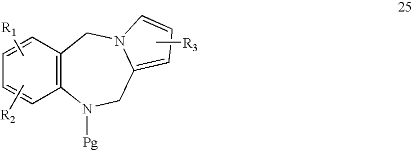
wherein

R₁, R₂ and R₃ are defined hereinbefore,

Pg is a protecting group;

with a an acid chloride under conditions sufficient to provide the desired intermediate of formula (26).

In some embodiments, the invention provides methods for preparing a compound of general formula II 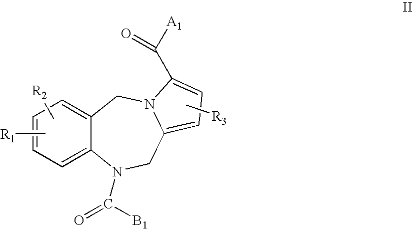
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, -OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];

R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B₁ is selected independently from the group consisting of 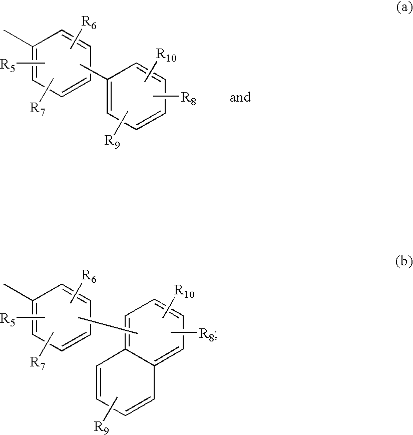
   wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxyalkyl, hydroxy(C₁-C₆) alkyl, alkyloxyalkyl, (C₁-C₆)alkoxy(C₁-C₆)alkyl, (C₂-C₇) acyloxy (C₁-C₆)alkyl, (C_{-C}₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, (C₁-C₆)alkyl substituted with 1-3 halogen atoms, trihalomethyl, trifluoromethyl, halogen, OCF₃, thioalkyl, thio(C₁-C₆) alkyl, —C(O) alkyl, —C(O)aryl optionally substituted by alkyl; hydroxy, —CH(OH)alkyl, —CH(alkoxy)alkyl, nitro, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₄, —(CH₂)ₚCONR₁₃R₁₄, —(CH₂)ₚCOOR₁₂, —CH═NOH, —CH═NO—(C₁-C₆) alkyl, trifluoromethylthio,

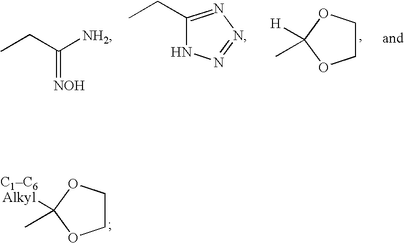

R₁₁ and R₁₂ are independently hydrogen or alkyl;

R₁₃ and R₁₄ are hydrogen or alkyl, or

R₁₃ and R₁₄ can be taken together with the nitrogen to which they are attached to form a 4-6 membered saturated ring optionally containing up to two ring heteroatoms selected from O, S or N;

p is 0 or 1;

A₁ is selected from the group consisting of

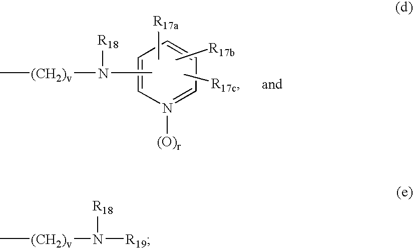

R₁₇ₐ, R_{17b}, and R_{17c} are each independently selected from the group consisting of hydrogen, alkyl, halogen, hydroxy, aryloxy, and hydroxyalkyl;

u is 0,1,2,3, or 4;

v is 1, 2, 3, or 4;

r is 0 or 1;

R₁₈ is hydrogen or alkyl; and

R₁₉ is a cycloalkylamine.

said method comprising:

reacting a compound of formula (2) 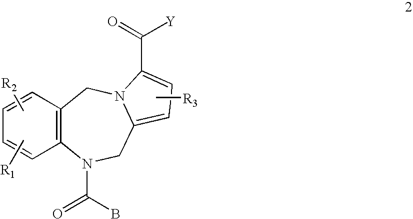
wherein Y is haloalkyl;
with an appropriate amine selected from 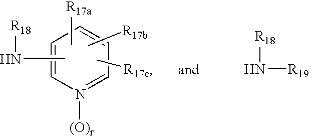
under conditions sufficient to produce the desired compound of formula II.

In some embodiments, the invention provides such methods where the compound of formula (2) is prepared by:

reacting a tricyclic diazepine of formula (1) 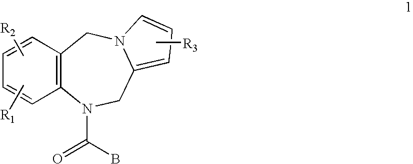
wherein R₁, R₂, and R₃ are defined hereinbefore,
with an acyl halide
XCOY
where X is a halide, and Y is haloalkyl;
under conditions sufficient to produce compound (2).

In some embodiments, the invention provides methods of preparing a compound according to formula III 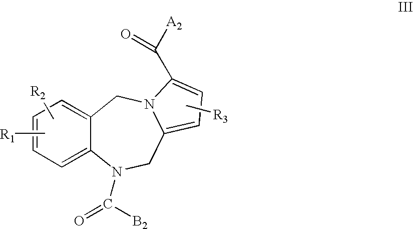
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl), —CON[(C₁-C₆) alkyl]₂, amino, (C₁-C₆) alkylamino, and —NHCO[(C₁-C₆) alkyl];

R₃ is a substituent selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B₂ is

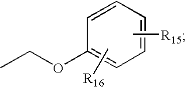

R₁₅ and R₁₆ are selected independently, from the group consisting of hydrogen, alkyl, and halogen;
and A₂ is selected from the group consisting of

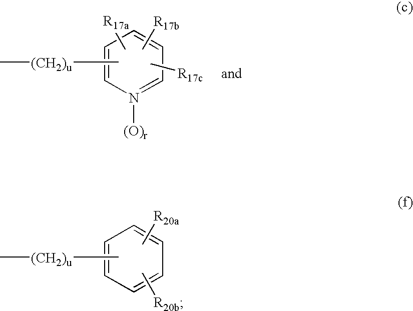

R₁₇ₐ, R_{17b}, and R_{17c} are each independently selected from the group consisting of hydrogen, alkyl, halogen, hydroxy, aryloxy, and hydroxyalkyl;

u is 0, 1, 2, 3, or 4;

r is 0 or 1;

R₂₀ₐ and R_{20b} are independently selected from the group consisting of hydrogen, alkyl, halogen, and aryl; or

R₂₀ₐ and R_{20b} can be taken together with the aryl to which they are attached to form a bicyclic system;

said method comprising:

reacting a tricyclkic diazepine of formula (5) 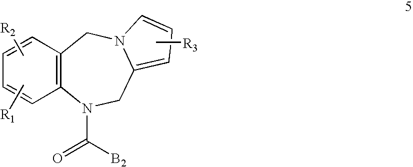
with an acid halide of formula 6
A₂COY (6)
wherein Y is halogen;
under conditions to produce a compound according to formula III.

In some embodiments, the invention provides methods for making a compound of formula (I) 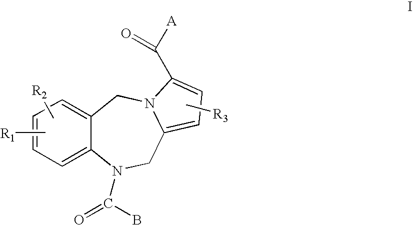
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];

R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B is B₁ or B₂,

wherein B₁ is selected independently from the group consisting of

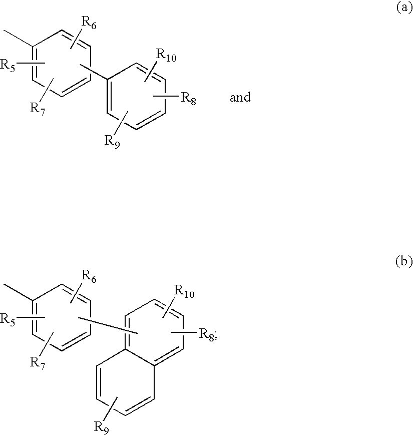

wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxy(C₁-C₆) alkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, halo (C₁-C₆)alkyl including trifluoromethyl, trihalomethyl, halogen, OCF₃, S((C₁-C₆) alkyl), —C(O) alkyl, —C(O)aryl optionally substituted by alkyl; hydroxy, hydroxyalkyl, alkyloxyalkyl, —CH(OH)alkyl, —CH(alkoxy)alkyl, formyl, nitro, thioalkyl, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCONR₁₃R₁₄, —(CH₂)ₚCOOR₁₂, —CH═NOH, —CH═NO—(C₁-C₆) alkyl, trifluoromethylthio, 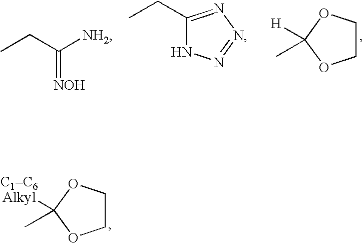
phenyl and naphthyl;

R₁₁ and R₁₂ are each independently hydrogen or alkyl;

R₁₃ and R₁₄ are each independently hydrogen or alkyl,

or R₁₃ and R₁₄ can be taken together with the nitrogen to which they are attached to form a 4-6 membered saturated ring optionally containing up to two atoms selected from O, S or N;

p is 0 or 1;
A is A₁ or A₂, wherein

A₁ is selected from

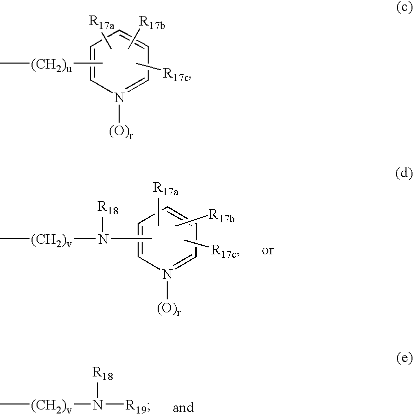

A₂ is selected from 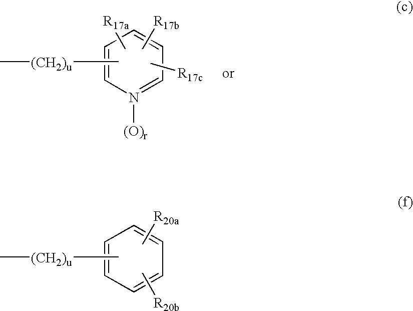
provided that when A is A₂, then B is B₂ wherein B₂ is

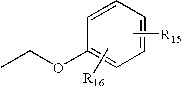

wherein R₁₅ and R₁₆ are selected independently from the group consisting of hydrogen, alkyl, and halogen;

wherein

R₁₇ₐ, R_{17b}, and R_{17c} are each independently selected from the group consisting of hydrogen, alkyl, halogen, hydroxy, aryloxy, and hydroxyalkyl;

u is the integer 0, 1, 2, 3, or 4;

v is the integer 1, 2, 3, or 4;

r is 0 or 1;

R₁₈ is hydrogen or alkyl; and

R₁₉ is a cycloalkylamine.

R₂₀ₐ and R_{20b} are each independently selected from the group consisting of hydrogen, alkyl, halogen, or aryl; or R₂₀ₐ and R_{20b} can be taken together with the aryl to which they are attached to form an aromatic bicycle having up to 10 total ring atoms;

said method comprising

treating a compound of formula (25) with an acid chloride of formula (4)
ACOY 4
under the conditions sufficient to yield the amide of formula (27) 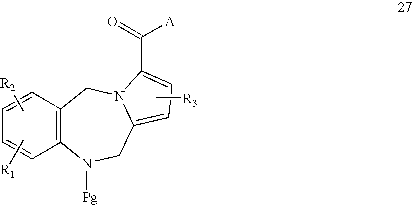
wherein A is A₂ as defined hereinbefore.

In some embodiments, the invention provides such methods further comprising deprotecting the compound of formula (27) to yield the intermediate of formula (28)

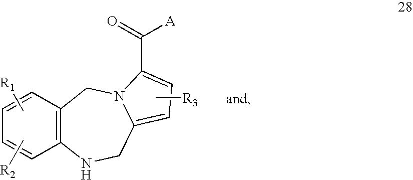

then acylating the intermediate of formula (28) to the desired product of formula (I).

In some embodiments, the invention provides the product made by any of the processes.

These and other embodiments will be recognized by those of skill in the art upon reading this specification.

### DETAILED DESCRIPTION OF THE INVENTION

In some embodiments, the invention provides compounds of formula (I): 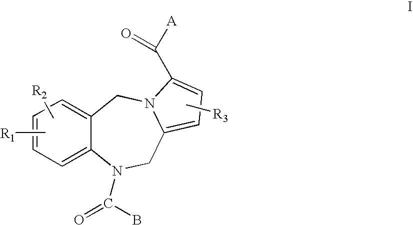
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, -OCF₃, carboxy, (C₁-C₆ alkoxy)carbonyl, —CONH₂, —CONH[(C₁-C₆) alkyl], —CON[(C₁-C₆) alkyl]₂ amino, (C₁-C₆) alkylamino, and —NHCO[(C₁-C₆) alkyl];

R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;

B is B₁ or B₂,

wherein B₁ is selected independently from the group consisting of

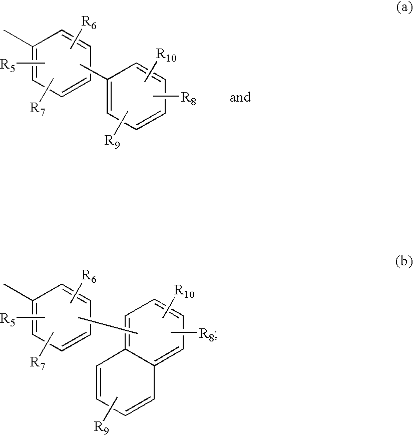

wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxyalkyl, hydroxy(C₁-C₆) alkyl, alkyloxyalkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈) cycloalkyl oxycarbonyl, aryl(C₁-C₆)alkyloxycarbonyl, carbamoyl, —O—CH₂—CH═CH₂, (C₁-C₆)alkyl substituted with 1-3 halogen atoms, trihalomethyl, trifluoromethyl, halogen, OCF₃, thioalkyl, thio(C₁-C₆) alkyl, —C(O) alkyl, —C(O)aryl optionally substituted by alkyl; hydroxy, —CH(OH)alkyl, —CH(alkoxy)alkyl, nitro, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCONR₁₃R₁₄, —(CH₂)ₚCOOR₁₂, —CH═NOH, —CH═NO—(C₁-C₆) alkyl, trifluoromethylthio,

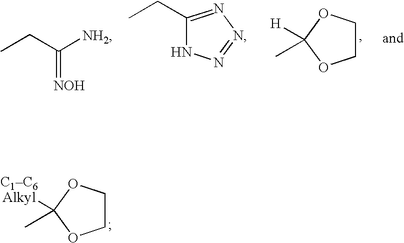

R₁₁ and R₁₂ are each independently hydrogen, alkyl, cycloalkyl, or C₃-C₈ cycloalkyl;

R₁₃ and R₁₄ are each independently hydrogen, alkyl, cycloalkyl, or C₃-C₈ cycloalkyl;

or R₁₃ and R₁₄ can be taken together with the nitrogen to which they are attached to form a 4-6 membered saturated ring optionally containing up to two atoms selected from O, S or N;

p is 0 or 1;

A is A₁ or A₂, wherein

A₁ is selected from

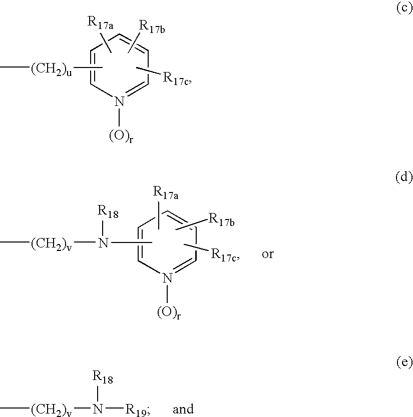

A₂ is selected from 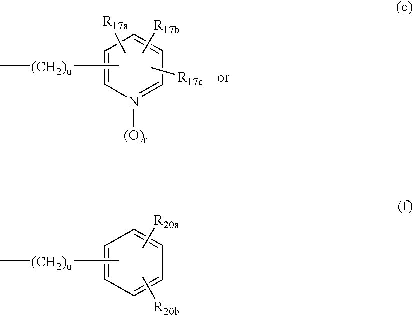
provided that when A is A₂, then B is B₂ wherein B₂ is

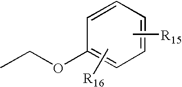

wherein R₁₅ and R₁₆ are selected independently from the group consisting of hydrogen, alkyl, C₁-C₆ alkyl, alkoxy, C₁-C₆ alkoxy, cyano, —CF₃, and halogen;

wherein

R₁₇ₐ, R_{17b}, and R_{17c} are each independently selected from the group consisting of hydrogen, alkyl, halogen, hydroxy, aryloxy, and hydroxyalkyl;

u is the integer 0, 1, 2, 3, or 4;

v is the integer 1, 2, 3, or 4;

r is 0 or 1;

R₁₈ is hydrogen, alkyl or C₁-C₆ alkyl; and

R₁₉ is a cycloalkylamine or a C₄-C₈ cycloalkylamine;

R₂₀ₐ and R_{20b} are each independently selected from the group consisting of hydrogen, alkyl, halogen, or aryl; or R₂₀ₐ and R_{20b} can be taken together with the aryl to which they are attached to form an aromatic bicycle having up to about 10 total ring atoms.

Other embodiments will be readily ascertainable to those of skill in the art upon reading this specification and claims.

Acyl, as used herein, refers to the group R—C(═O)— where R is an alkyl group of 1 to 6 carbon atoms. For example, a C₂ to C₇ acyl group refers to the group R—C(═O)— where R is an alkyl group of 1 to 6 carbon atoms.

Alkenyl, as used herein, refers to an alkyl group having one or more double carbon-carbon bonds. Example alkenyl groups include, but are not limited to, ethenyl, propenyl, butenyl, pentenyl, hexenyl, butadienyl, pentadienyl, hexadienyl, and the like. In some embodiments, alkenyl groups can be substituted with up to four substituent groups, as described below.

Alkoxy, as used herein, refers to an —O-alkyl group. Example alkoxy groups include, but are not limited to, methoxy, ethoxy, propoxy (e.g., n-propoxy and isopropoxy), t-butoxy, and the like. An alkoxy group can contain from 1 to about 20, 1 to about 10, 1 to about 8, 1 to about 6, 1 to about 4, or 1 to about 3 carbon atoms. In some embodiments, alkoxy groups can be substituted with up to four substituent groups.

Alkoxyalkyl, employed alone or in combination with other terms, refers to an alkoxy, as herein before defined, which is further covalently bonded to an unsubstituted (C₁-C₁₀) straight chain or unsubstituted (C₂-C₁₀) branched-chain hydrocarbon. Examples of alkoxyalkyl moieties include, but are not limited to, chemical groups such as, but not limited to, methoxymethyl, —CH₂CH(CH₃)OCH₂CH₃, and homologs, isomers, and the like.

Alkoxycarbonyl, employed alone or in combination with other terms, is defined herein as, unless otherwise stated, an alkoxy group, as herein before defined, which is further bonded to a carbonyl group to form an ester moiety. Examples of alkoxycarbonyl moieties include, but are not limited to, chemical groups such as, but not limited to, methoxycarbonyl, ethoxycarbonyl, isopropoxycarbonyl, sec-butoxycarbonyl, tert-butoxycarbonyl, decanoxycarbonyl, and homologs, isomers, and the like.

Alkyl refers to a saturated hydrocarbon group which is straight-chained or branched. Example alkyl groups include, but are not limited to, methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. Alkyl groups can contain from 1 to about 20, 1 to about 10, 1 to about 8, 1 to about 6, 1 to about 4, or 1 to about 3 carbon atoms. In some embodiments, alkyl groups can be substituted with up to four substituent groups, as described below. Lower alkyl is intended to mean alkyl groups having up to six carbon atoms.

Alkylamino, employed alone or in combination with other terms, refers to a moiety with one alkyl group, wherein the alkyl group is an unsubstitued (C₁-C₆) straight chain hereunto before defined alkyl group or an unsubstitued (C₃-C₈) hereunto before defined cycloalkyl group. Examples of alkylamino moieties include, but are not limited to, chemical groups such as, but not limited to, —NH(CH₃), —NH(CH₂CH₃), —NH-cyclopentyl, and homologs, and the like.

Alkylaminosulfonyl refers to an alkylamino moiety, as herein before defined, which is further bonded to a sulfonyl group.

Alkylsulfonyl, as used herein, refers to the group R—S(O)₂— where R is an alkyl group.

Alkynyl, as used herein, refers to an alkyl group having one or more triple carbon-carbon bonds. Examples of alkynyl groups include, but are not limited to, ethynyl, propynyl, butynyl, pentynyl, and the like. In some embodiments, alkynyl groups can be substituted with up to four substituent groups, as described below.

Aroyl, as used herein, refers to the group Ar—C(═O)— where Ar is aryl as defined above. For example, a C₆ to C₁₄ aroyl moiety refers to the group Ar—C(═O)— where Ar is an aromatic 5 to 13 membered carbocylic ring.

Aryl, as used herein, refers to aromatic carbocyclic groups including monocyclic or polycyclic aromatic hydrocarbons such as, but not limited to, for example, phenyl, 1-naphthyl, 2-naphthyl anthracenyl, phenanthrenyl, and the like. In some embodiments, aryl groups have from 5 to about 20 carbon atoms. In some preferred embodiments, aryl groups are phenyl or naphthyl groups that optionally contain up to four, preferably up to 2, substituent groups as described below.

Arylalkyl or aralkyl, as used herein, refers to a group of formula -alkyl-aryl. Preferably, the alkyl portion of the arylalkyl group is a lower alkyl group, i.e., a C₁-C₆ alkyl group, more preferably a C₁-C₆ alkyl group. Examples of aralkyl groups include, but are not limited to, benzyl and naphthylmethyl groups. In some preferred embodiments, arylalkyl groups can be optionally substituted with up to four, preferably up to 2, substituent groups.

Aryloxy, as used herein, refers to an —O-aryl group, for example and not limitation, phenoxy.

Bicyclic system, as used herein, refers to a saturated, partially saturated, or aromatic bicycle having 6-20 total ring atoms, preferably 8-12 total ring atoms, and most preferably 10 total ring atoms, and from 0-3 ring heteroatom selected from O, S, and N, preferably with 1 ring heteroatom. Exemplary bicyclic systems include, but are not limited to, napthyl, quinoline, and isoquinoline.

Carbamoyl, as used herein, refers to the group, —C(═O)N<.

Carbonyl, employed alone or in combination with other terms, refers to a bivalent one-carbon moiety further bonded to an oxygen atom with a double bond. An example is

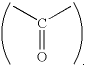

Carboxy as employed herein refers to —COOH.

Cyano, as used herein, refers to CN.

Cycloalkyl, as used herein, refers to non-aromatic carbocyclic groups including cyclized alkyl, alkenyl, and alkynyl groups. Cycloalkyl groups can be monocyclic (e.g., cyclohexyl) or poly-cyclic (e.g. 2, 3, or 4 fused ring) ring systems. Examples of cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of cyclopentane (indanyl), cyclohexane (tetrahydronaphthyl), and the like.

Cycloalkylalkyl, as used herein, refers to a group of formula -alkyl-cycloalkyl, for example a cyclopropylmethyl group.

Cycloalkylcarbonyl, as used herein, refers to a group of formula -carbonyl-cycloalkyl, for example cyclohexylcarbonyl.

Dialkylamino, employed alone or in combination with other terms, refers to a moiety with two independent alkyl groups, wherein the alkyl groups are unsubstitued (C₁-C₆) straight chain hereunto before defined alkyl groups or unsubstitued (C₃-C₈) hereunto before defined cycloalkyl groups. The two groups may be linked to form an unsubstituted (C₁-C₆)-alkylene-group. Examples of dialkylamino moieties include, but are not limited to, chemical groups such as, but not limited to, —N(CH₃)₂, —N(CH₂CH₃)₂, —NCH₃(CH₂CH₃), 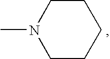
and homologs, and the like.

Dialkylaminoalkyl, employed alone or in combination with other terms, refers to a dialkylamino moiety, as herein before defined, which is further covalently bonded to a straight chain alkyl group of 1-6 carbon atoms. Examples of dialkylaminoalkyl moieties include, but are not limited to, chemical groups such as, but not limited to, —CH₂N(CH₃)₂, —CH₂CH₂N(CH₂CH₃)₂, —CH₂CH₂CH₂NCH₃(CH₂CH₃), and homologs, and the like.

Halo or halogen includes fluoro, chloro, bromo, and iodo.

Hünig's Base is N,N-diisopropylethylamine, also indicated herein as i-Pr₂NEt.

Hydroxy or hydroxyl, as used herein, refers to OH.

Hydroxyalkyl, employed alone or in combination with other terms, refers to a (C₁-C₁₀) straight chain hydrocarbon, terminally substituted with a hydroxyl group. Examples of hydroxyalkyl moieties include, but are not limited to, chemical groups such as, but not limited to, —CH₂OH, —CH₂CH₂OH, —CH₂CH₂CH₂OH, and higher homologs.

Nitro, employed alone or in combination with other terms, is defined herein as, —NO₂.

Thioalkyl, employed alone or in combination with other terms, is defined herein as sulfur covalently bonded via a double bond to an alkyl group as defined above.

Substituted, as used herein, refers to a moiety, such as, but not limited to, an aryl or heteroaryl, having from 1 to about 5 substituents, and more preferably from 1 to about 3 substituents independently selected from a halogen atom, a cyano group, a nitro group, a hydroxyl group, a C₁-C₆ alkyl group, or a C₁-C₆ alkoxy group. Preferred substituents are a halogen atom, a hydroxyl group, or a C₁-C₆ alkyl group.

At various places in the specification, substituents of compounds of the invention are disclosed in groups or in ranges. It is specifically intended that the invention include each and every individual subcombination of the members of such groups and ranges. For example, the term C₁-C₆ alkyl is specifically intended to individually disclose methyl, ethyl, propyl, isopropyl, n-butyl, sec-butyl, isobutyl, etc.

In some embodiments, the invention provides such a compound wherein A is A₁.

In some embodiments, A₁ is

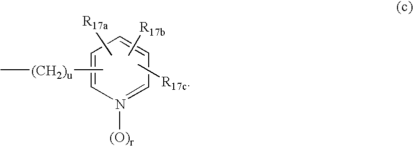

In some embodiments, A₁ is

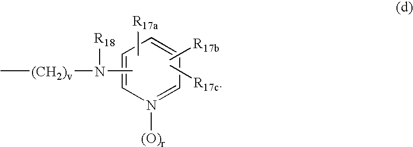

In some embodiments, A₁ is

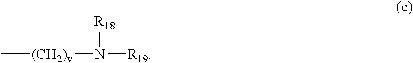

In some embodiments, B is B₁, and B₁ is

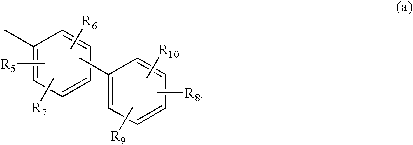

In some embodiments, B is B₁, and B₁ is

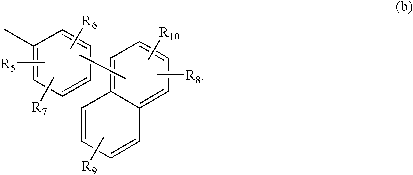

In some embodiments, the invention provides compounds of formula I wherein A is A₂ and B is B₂.

In some such embodiments A₂ is

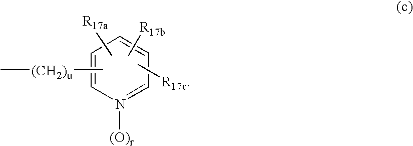

In other such embodiments, A₂ is

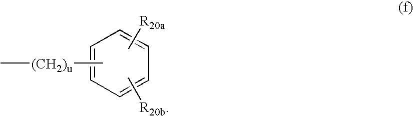

In some embodiments, the invention provides compounds represented by the formula II 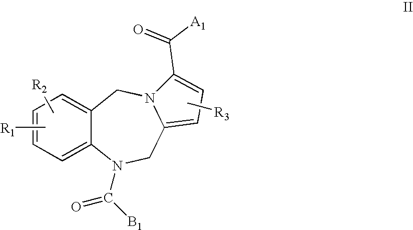
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];

R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;

B₁ is selected independently from the group consisting of 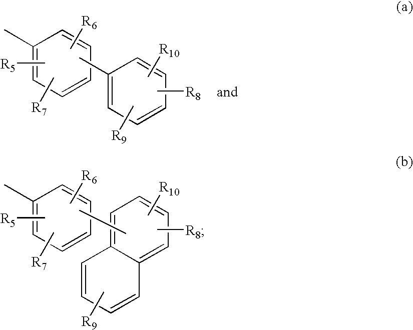
wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxyalkyl, hydroxy(C₁-C₆) alkyl, alkyloxyalkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, (C₁-C₆)alkyl substituted with 1-3 halogen atoms, trihalomethyl, trifluoromethyl, halogen, OCF₃, thioalkyl, thio(C₁-C₆) alkyl, —C(O) alkyl, —C(O)aryl optionally substituted by alkyl; hydroxy, —CH(OH)alkyl, —CH(alkoxy)alkyl, nitro, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCON R₁₃R₁₄, —(CH₂)ₚCOOR₁₂, —CH═NOH, —CH═NO—(C₁-C₆) alkyl, trifluoromethylthio,

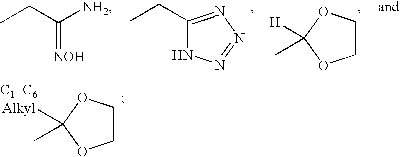

R₁₁ and R₁₂ are each independently hydrogen, alkyl, cycloalkyl, or C₃-C₈ cycloalkyl;

R₁₃ and R₁₄ are each independently hydrogen, alkyl, cycloalkyl, or C₃-C₈ cycloalkyl;

or R₁₃ and R₁₄ can be taken together with the nitrogen to which they are attached to form a 4-6 membered saturated ring optionally containing up to two atoms selected from O, S or N;

p is 0 or 1;
A₁ is selected from the group consisting of

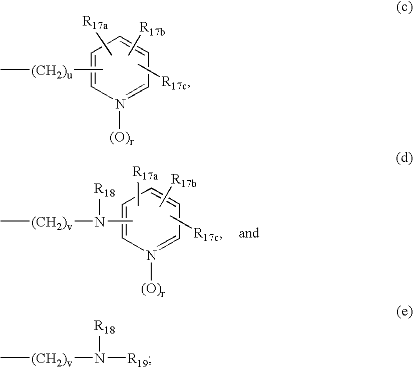

R₁₇ₐ, R_{17b}, and R_{17c} are each independently selected from the group consisting of hydrogen, alkyl, halogen, hydroxy, aryloxy, and hydroxyalkyl;

u is 0, 1, 2, 3, or 4;

v is 1, 2, 3, or 4;

r is 0 or 1;

R₁₈ is hydrogen or alkyl; and

R₁₉ is a cycloalkylamine.
In some embodiments, the invention provides such compounds of formula II, wherein A₁ is 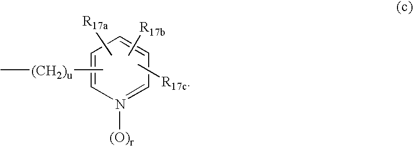
In some embodiments, the invention provides such compounds of formula II, wherein u is 2.
In some embodiments, the invention provides such compounds of formula II, wherein r is 0.
In some embodiments, the invention provides such compounds of formula II, wherein A₁ is

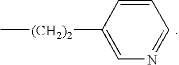

In some embodiments, the invention provides such compounds of formula II, wherein B₁ is 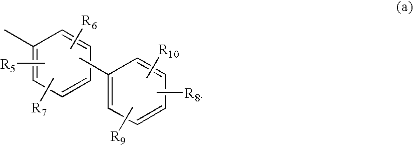
In some such embodiments, each of R₅-R₁₀ is hydrogen. In some embodiments, one of R₈-R₁₀ is alkyl, in some preferred embodiments, one of R₈-R₁₀ is methyl.

In other embodiments, B₁ is 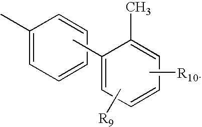
In some such embodiments, one of R₈-R₁₀ is alkoxy, preferably, one of said R₈-R₁₀ is methoxy.

In other embodiments, B₁ is

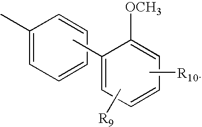

In some embodiments the invention provides compounds of formula II where A₁ is

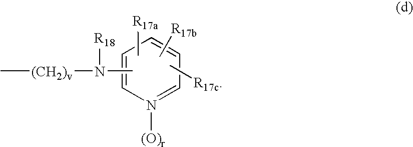

In some such embodiments, v is 1. In others, r is 0. In yet other embodiments, v is 1 and r is 0. In some such embodiments, the ring nitrogen is in the 3-position.
A compound of formula II where A₁ is 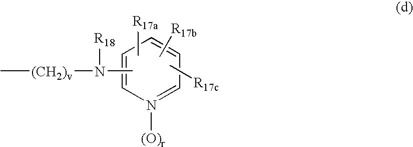
   and B₁ is 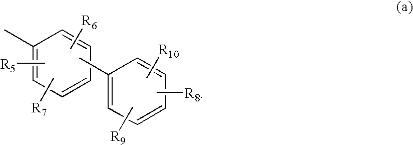
In some such embodiments, each of R₅-R₁₀ is hydrogen. In some embodiments, one of R₈-R₁₀ is alkyl, preferably one of said R₈-R₁₀ is methyl.
In some embodiments, the invention provides a compound of formula II wherein A₁ is

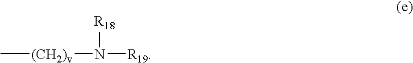

Other embodiments of the invention provide compounds represented by the formula III 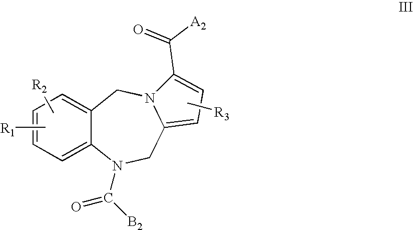
or a pharmaceutically acceptable salt thereof,
wherein

R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];

R₃ is a substituent selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;

B₂ is

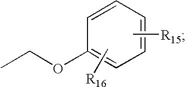

R₁₅ and R₁₆ are selected independently, from the group consisting of hydrogen, alkyl, and halogen;
and A₂ is selected from the group consisting of

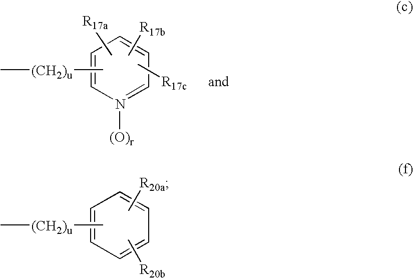

R₁₇ₐ, R_{17b}, and R_{17c} are each independently selected from the group consisting of hydrogen, alkyl, halogen, hydroxy, aryloxy, and hydroxyalkyl;

u is 0, 1, 2, 3, or 4;

r is 0 or 1;

R₂₀ₐ and R_{20b} are independently selected from the group consisting of hydrogen, alkyl, halogen, and aryl; or

R₂₀ₐ and R_{20b} can be taken together with the aryl to which they are attached to form a bicyclic system such as, but not limited to, a naphthyl.

In some such embodiments the invention provides compounds of formula III wherein A₂ is 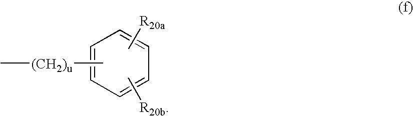
In some such embodiments, u is 0. In some such embodiments, R₂₀ₐ is halogen, preferably chlorine.

In some embodiments the invention provides compounds of formula III wherein R₂₀ₐ and R_{20b} taken together with the aryl to which they are attached to form a bicyclic structure. In some embodiments, the bicyclic structure is naphthalene.

In some embodiments the invention provides compounds of formula III wherein wherein R₂₀ₐ is aryl, preferably phenyl.

In some embodiments the invention provides compounds of formula III where A₂ is

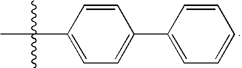

In some embodiments the invention provides compounds of formula III wherein A₂ is

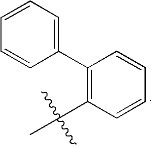

In some embodiments the invention provides compounds of formula III wherein R₂₀ₐ is alkyl, particularly C(CH₃)₃.

In some embodiments the invention provides compounds of formula III wherein A₂ is

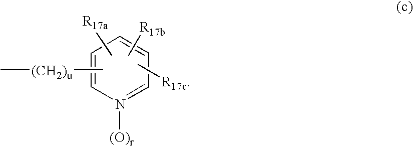

In some such embodiments B₂ is

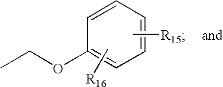

one of R₁₅ or R₁₆ is halogen, particularly chlorine. In some such embodiments, the other one of R₁₅ or R₁₆ is alkyl, particularly methyl. In some preferred embodiments, R₁₅ is 4-chloro and R₁₆ is 2-methyl.

Some exemplary compounds include, but are not limited to, those in the following table:

| Ex- | |
|---|---|
| am- | |
| ple | Structure |
| 1 | 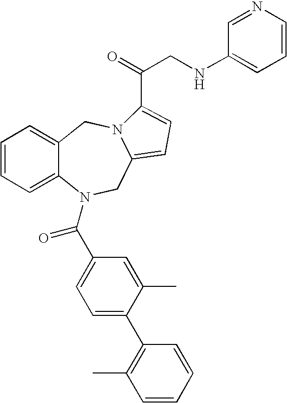 |
| 2 | 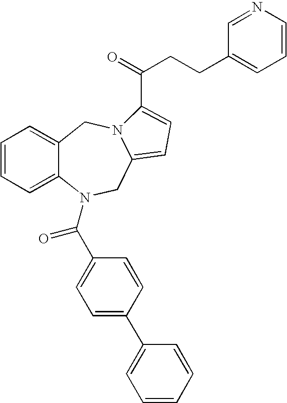 |
| 3 | 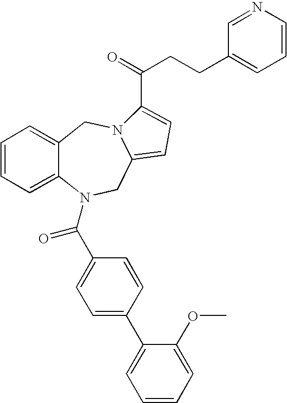 |
| 4 | 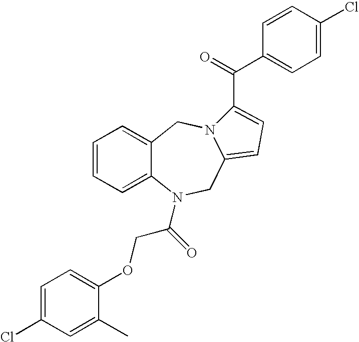 |
| 5 | 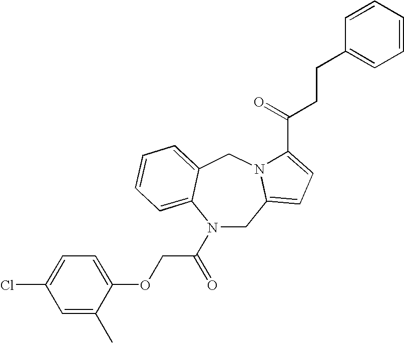 |
| 6 | 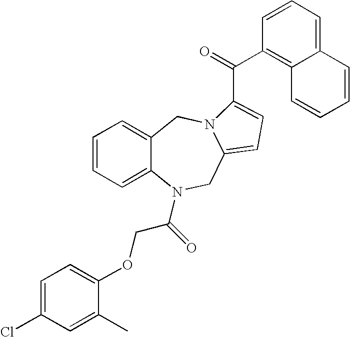 |
| 7 | 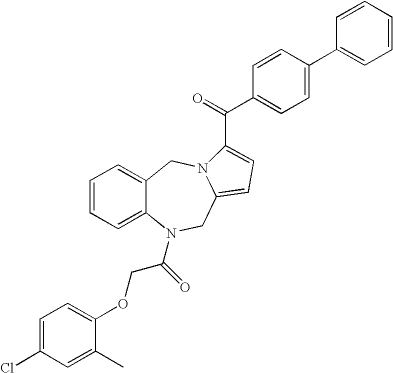 |
| 8 | 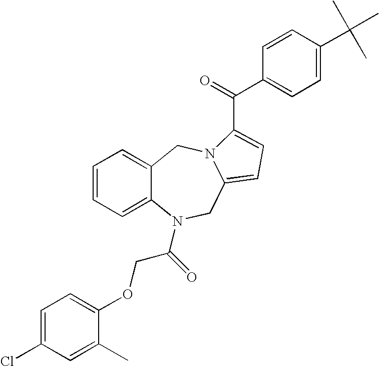 |
| 9 | 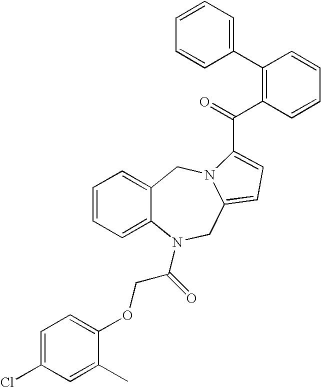 |
| 10 | 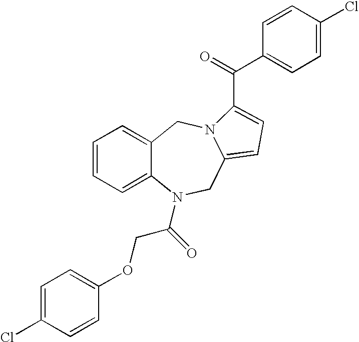 |
| 11 | 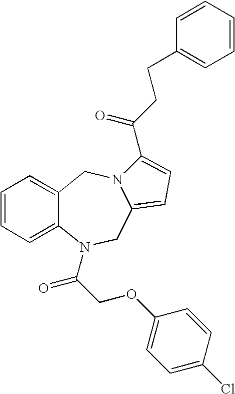 |
| 12 | 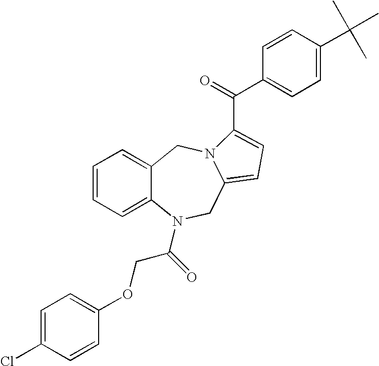 |
| 13 | 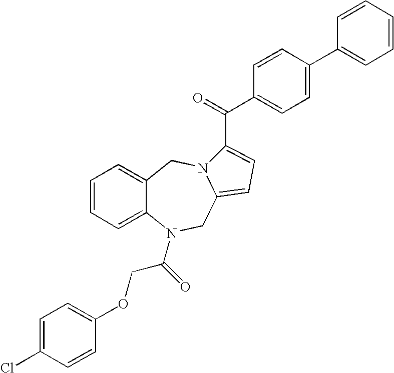 |
| 14 | 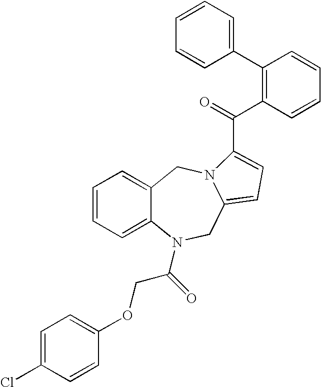 |
| 15 | 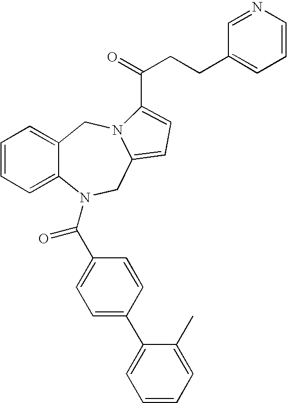 |

Those practicing the art will readily recognize that some of the compounds of this invention, depending on the definition of the various substituents, can contain one or more asymmetric centers, and can give rise to enantiomers and diastereomers. The present invention includes all stereoisomers including individual diastereomers and resolved, enantiomerically pure R and S stereoisomers; as well as racemates, and all other mixtures of R and S stereoisomers and pharmaceutically acceptable salts thereof, which possess the indicated activity. Optical isomers may be obtained in pure form by standard procedures known to those skilled in the art. It is also understood that this invention encompasses all possible regioisomers, E-Z isomers, endo-exo isomers, and mixtures thereof which posses the indicated activity. Such isomers can be obtained in pure form by standard procedures known to those skilled in the art.

Those practicing the art will readily recognize that some of the compounds of this invention, depending on the definition of various subsituents, may be chiral due to hindered rotation, and give rise to atropisomers which can be resolved and obtained in pure form by standard procedures known to those skilled in the art. Also included in this invention are all polymorphs and hydrates of the compounds of the present invention.

Some embodiments of the invention also includes pharmaceutically acceptable salts of the compounds disclosed herein. By “pharmaceutically acceptable salt”, it is meant any compound formed by the addition of a pharmaceutically acceptable base and a compound disclosed herein to form the corresponding salt. By the term “pharmaceutically acceptable” it is meant a substance that is acceptable for use in pharmaceutical applications from a toxicological perspective and does not adversely interact with the active ingredient. Pharmaceutically acceptable salts, including mono- and bi-salts, include, but are not limited to, those derived from such organic and inorganic acids such as, but not limited to, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids.

Methods

### General Synthetic Schemes for Preparation of Compounds

The compounds of the present invention may be prepared according to one or more of the general processes outlined below.

The compounds of general formula (II) wherein B is B₁ which is 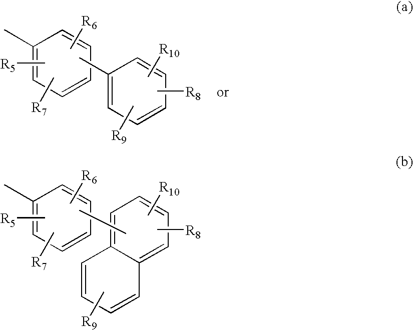
A₁ is 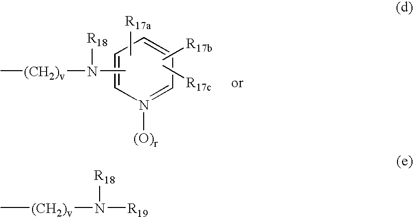
can be conveniently prepared as shown in Scheme I.

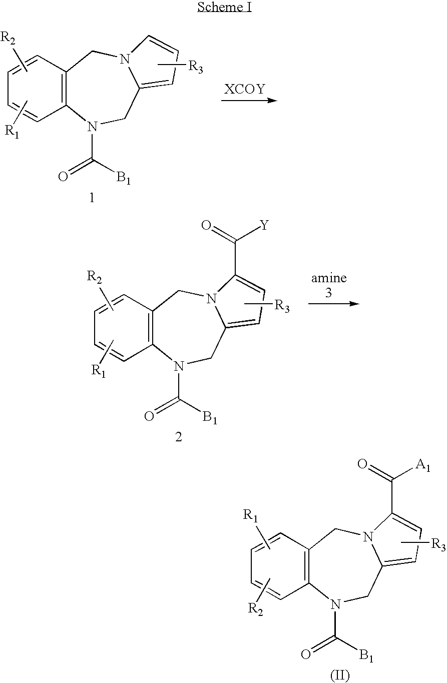

According to the above preferred process, a tricyclic diazepine of formula (1) wherein R₁, R₂, and R₃ are defined hereinbefore, is reacted with an acyl halide preferably an acid chloride where X is Cl in an aprotic organic solvent such as, but not limited to, 1,4-dioxane at temperatures ranging from −10° C. to reflux, to provide the desired intermediate of formula (2) where Y is haloalkyl, preferably chloroalkyl. Subsequent reaction of the intermediate of formula (2) with an appropriate amine of formula (3) at temperatures ranging from ambient to the refluxing temperature of the solvent or in the absence of a solvent to the melting point of the reactants, provides the desired compounds of formula (II) wherein R₁, R₂, R₃, and A₁ are as defined hereinbefore. When the amine of formula (3) is an appropriately substituted pyridylamine or a dialkylamine. The compounds of formula (1) can be further converted to their N-oxides by treatment with an oxidizing agent such as, but not limited to, a peracid or other pyridine oxidizing agents known in the literature at temperatures ranging from −40° C. to ambient temperature.

A preferred process for preparing compounds of general formula (II) wherein B is B₁ which is 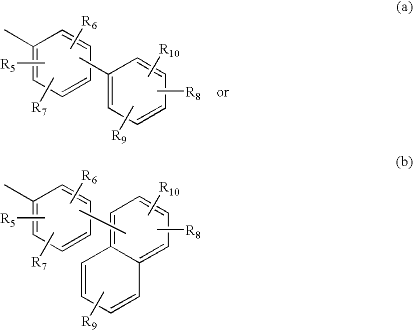
and A₁ is 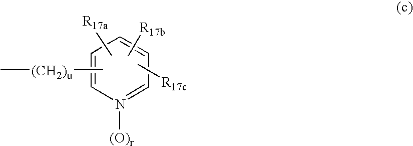
is shown in Scheme II below.

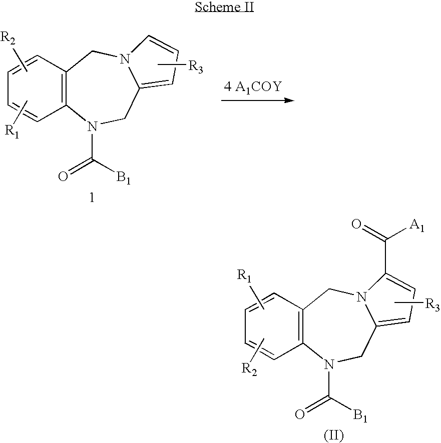

Thus, a tricyclic diazepine of formula (1) wherein R₁, R₂, and R₃ are defined hereinbefore, is reacted with an acyl halide, preferably an acid chloride of formula (4), wherein Y is Cl, either in the presence of an aprotic organic solvent such as, but not limited to, N-methyl-2-pyrrolidinone at temperatures ranging from ambient to reflux, or in the absence of a solvent to the melting point of the reactants, and in the presence or absence of an organic base such as, but not limited to, 2,6-lutidine, to provide the desired compounds of formula (II) wherein R₁, R₂, R₃, and A₁ are as defined hereinbefore. The compounds of formula (II) of Scheme II can be further converted to their N-oxides by treatment with an oxidizing agent such as, but not limited to, a peracid or other pyridine oxidizing agents known in the literature at temperatures ranging from −40° C. to ambient temperature.

The compounds of formula (III) wherein R₁, R₂, R₃, A₂ and B₂ are defined hereinbefore, can be prepared as shown in Scheme III by reacting a tricyclic diazepine of formula (5) with an acid halide, preferably an acid chloride of formula (4), where Y is Cl under the conditions of Scheme II.

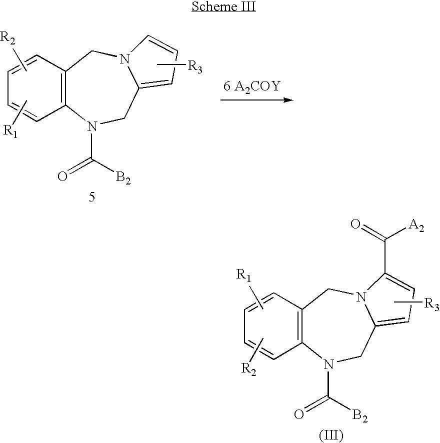

The compounds of formula (III) of Scheme III wherein A₂ contains a pyridine moiety can be further converted to their N-oxides by treatment with an oxidizing agent such as, but not limited to, a peracid or other pyridine oxidizing agents known in the literature at temperatures ranging from −40° C. to ambient temperature.

The tricyclic diazepines of formula (1) of Scheme I wherein B is B₁ which is 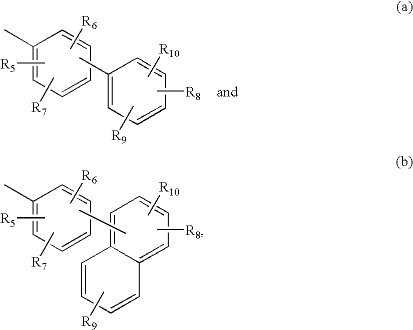
can be conveniently prepared as shown in Scheme IV

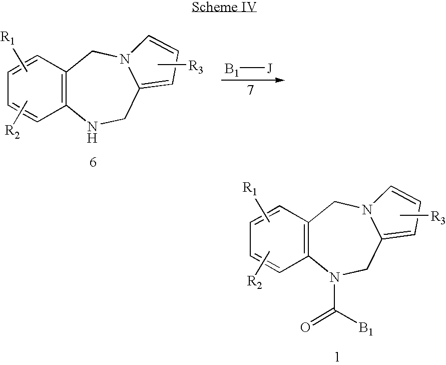

Thus, a tricyclic diazepine of formula (6) is treated with an appropriately substituted acylating agent, preferably an appropriately substituted acyl chloride or acyl bromide of formula (7), where J is COCl or COBr, respectively, in the presence of an inorganic base such as, but not limited to, potassium carbonate, or in the presence of an organic base such as, but not limited to, pyridine, 4-(dimethylamino)pyridine, or a tertiary amine such as, but not limited to, triethylamine, N,N-diisopropylethyl amine or N,N-dimethylaniline, in an aprotic solvent such as, but not limited to, dichloromethane, N,N-dimethylformamide, tetrahydrofuran or 1,4-dioxane, at temperatures ranging from −5° C. to 50° C. to provide intermediates of general formula (1) wherein B₁ is defined hereinbefore.

Alternatively, the acylating species of formula (7) can be a mixed anhydride of the corresponding carboxylic acid, such as, but not limited to, that prepared by treating said acid with 2,4,6-trichlorobenzoyl chloride in an aprotic organic solvent such as, but not limited to, dichloromethane according to the procedure of Inanaga et al., Bull. Chem. Soc. Jpn., 52, 1989 (1979). Treatment of said mixed anhydride of general formula (7) with a tricyclic diazepine of formula (6) in a solvent such as, but not limited to, dichloromethane, and in the presence of an organic base such as, but not limited to, 4-(dimethylaminopyridine), at temperatures ranging from 0° C. to the reflux temperature of the solvent, yields the intermediate acylated derivative (1) of Scheme IV.

The acylating intermediate of formula (7) is ultimately chosen on the basis of its compatibility with B groups, and its reactivity with the tricyclic diazepine of formula (6).

The desired intermediates of formula (7) of Scheme IV wherein B is B₁ and B₁ is 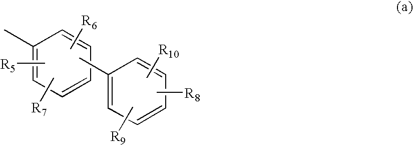
can be conveniently prepared by a process shown in Scheme V. Thus, an appropriately substituted aryl iodide, aryl bromide, aryl chloride, or aryl trifluoromethane sulfonate of formula (8), wherein Pg is a carboxylic acid protecting group, preferably Pg is alkyl or benzyl, M is I, Br, Cl, or OTf, and R₅, R₆ and R₇ are defined hereinbefore, is reacted with an aryl tri(alkyl)tin(IV) derivative of formula (9), where T is Sn(alkyl)₃, preferably Sn(n-Bu)₃, and wherein R₈, R₉ and R₁₀ are defined hereinbefore, in the presence of a Pd(0) catalyst, in the presence or absence of inorganic salts (e.g. LiCl or copper(I) salts), to provide the intermediate ester of formula (10). Subsequent unmasking of the carboxylic function by hydrolysis, hydrogenolysis or similar methods known in the art, followed by activation of the intermediate acid of formula (11) provides the desired compounds of formula (7) wherein R₅, R₆, R₇, R₈, R₉ and R₁₀ are hereinbefore defined, suitable for coupling with the tricyclic diazepine of formula (6).

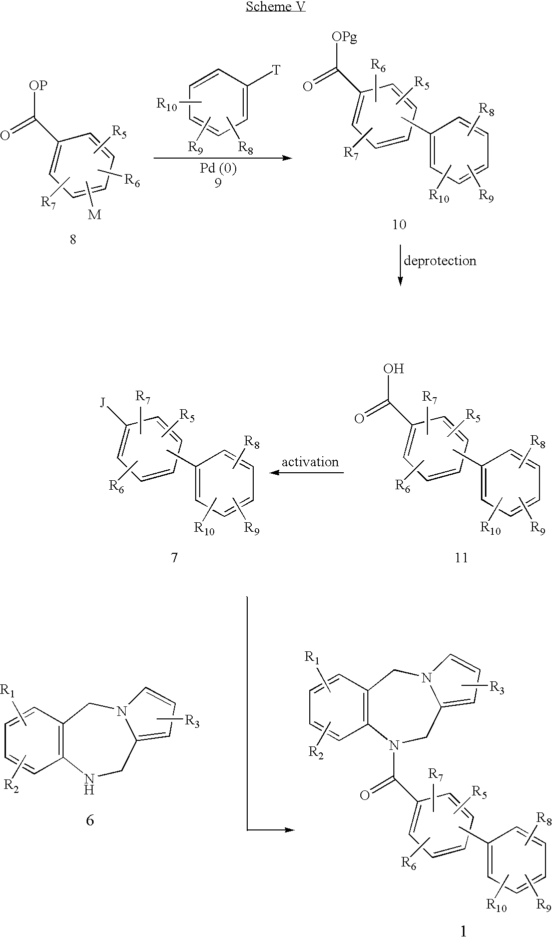

The desired intermediates of formula (7) of Scheme IV wherein B is B₁ and B₁ is 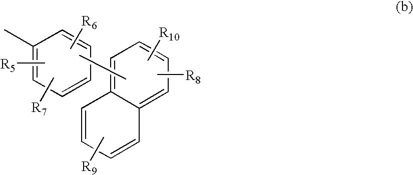
can be prepared by a process analogous to that exemplified in Scheme V by replacing intermediates of formula (9) with appropriately substituted naphthyl intermediates.

Alternatively, the desired intermediates of formula (10) of Scheme V wherein B is B₁ and B₁ is 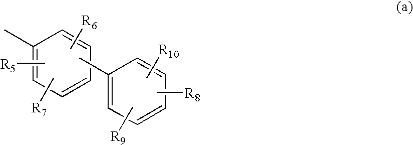
can be prepared by the coupling of the intermediate of formula (8) where M is I, Br, Cl or OTf, and an appropriately substituted aryl boron derivative of formula (9), preferably where T is B(OH)₂, in the presence of a palladium catalyst such as, but not limited to, palladium(II) acetate or tetrakis(triphenylphosphine) palladium(0) and an organic base such as, but not limited to, triethylamine or an inorganic base such as, but not limited to, sodium carbonate, potassium carbonate, or cesium carbonate with or without added tetrabutylammonium bromide or tetrabutylammonium iodide, in a mixture of solvents such as, but not limited to, toluene-ethanol-water, acetone-water, water or water-acetonitrile, at temperatures ranging from ambient to the reflux temperature of the solvent (Suzuki, Pure & Appl. Chem. 66, 213-222 (1994), Badone et al., J. Org. Chem. 62, 7170-7173 (1997), Wolfe et al. J. Am. Chem. Soc. 121, 9559 (1999), Shen, Tetr. Letters 38, 5575 (1997)). The exact conditions for the Suzuki coupling of the halide and the boronic acid intermediates are chosen on the basis of the nature of the substrate and the substituents. The desired intermediates of formula (10) of Scheme V can be similarly prepared from the bromide of formula (8), where M is Br, and the boronic acid of formula (9) in a solvent such as, but not limited to, dioxane in the presence of potassium phosphate and a Pd(0) catalyst.

Alternatively, a palladium-catalyzed cross-coupling reaction of an aryl halide (or trifluoromethane sulfonate) of formula (9), where T is Br, I or OTf, with a pinacolato boronate, or boronic acid or trialkyltin(IV) derivative of formula (8), where M is 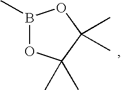
B(OH)₂, or SnBu₃, yields the desired intermediate of formula (10) which is converted to a compound of formula (1) in the manner of Scheme V.

The desired intermediates of formula (10) of Scheme V wherein B is B₁ and B₁ is 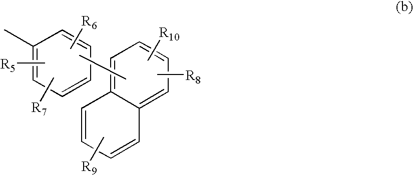
can be prepared in analogous fashion by replacing intermediates of formula (9) with appropriately substituted naphthyl intermediates.

The required appropriately substituted aryl halides of formula (8), where M is Br or I, of Scheme V are either available commercially, or are known in the art, or can be readily accessed in quantitative yields and high purity by diazotization of the corresponding substituted anilines of formula (8), where Pg is H, alkyl or benzyl, and M is NH₂, followed by reaction of the intermediate diazonium salt with iodine and potassium iodide in aqueous acidic medium essentially according to the procedures of Street et al,. J. Med. Chem. 36, 1529 (1993) and Coffen et al., J. Org. Chem. 49, 296 (1984) or with copper(I) bromide, respectively (March, Advanced Organic Chemistry, 3^{rd} Edn., p. 647-648, John Wiley & Sons, New York (1985)).

Alternatively, the desired intermediates of formula (11) of Scheme V wherein B is B₁ and B₁ is 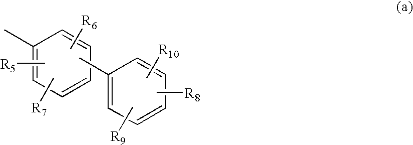
can be conveniently prepared as shown in Scheme VI by cross-coupling reaction of an appropriately substituted pinacolato boronate of formula (13) wherein R₈, R₉ and R₁₀ are hereinbefore defined, with an aryl triflate or an aryl halide of formula (14), where W is OTf, Br, I) wherein R₅, R₆ and R₇ are defined hereinbefore, according to the general procedures of Ishiyama et al., Tetr. Lett. 38, 3447-3450 (1997) and Giroux et al. Tetr. Lett. 38, 3841-3844 (1997), followed by basic or acidic hydrolysis of the intermediate nitrile of formula (15) (cf. March, Advanced Organic Chemistry, 3^{rd} Edn., John Wiley & Sons, New York, p. 788 (1985)).

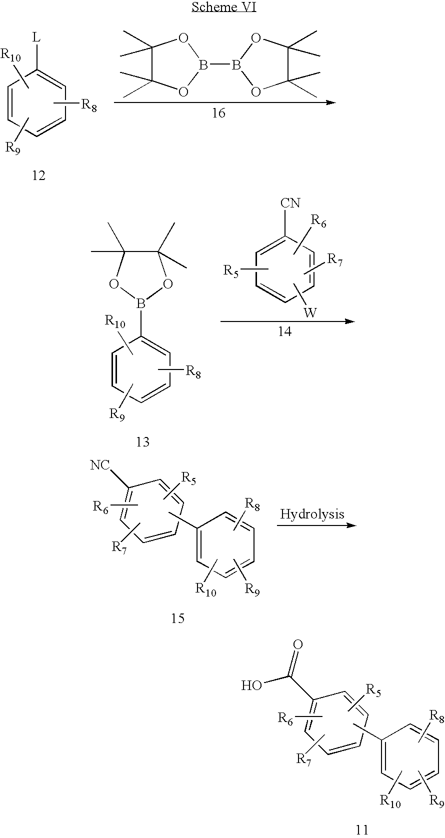

Alternatively, reaction of an intermediate of formula (12), where L is Br, Cl, I, or OTf with a derivative of formula (13), where W is B(OH)₂, or SnBu₃, yields the desired intermediate of formula (15) which is converted to intermediate (11) in the manner of Scheme VI.

The desired intermediates of formula (15) of Scheme VI where B is B₁ and B₁ is 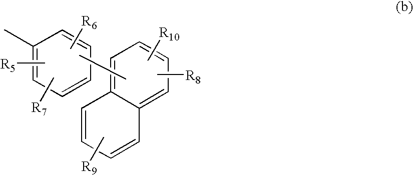
can be prepared in analogous fashion by replacing intermediates of formula (13) with appropriately substituted naphthyl intermediates.

The desired phenyl boronic esters of formula (13) of Scheme VI can be conveniently prepared by the palladium-catalyzed cross-coupling reaction of bis(pinacolato)diboron of formula (16) with an appropriately substituted aryl halide or aryl triflate of formula (12), where L is OTf. In preferred aryl halides of formula (12) L is Br, or I. The reaction is carried out according to the described procedures of Ishiyama et al., J. Org. Chem. 60, 7508-7510 (1995) and Giroux et al., Tetr. Lett. 38, 3841-3844 (1997).

The desired compounds of formula (1) of Scheme IV wherein B is B₁ and B₁ is 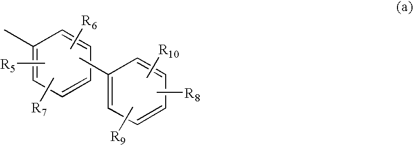
can be alternatively prepared by a process shown in Scheme VII. 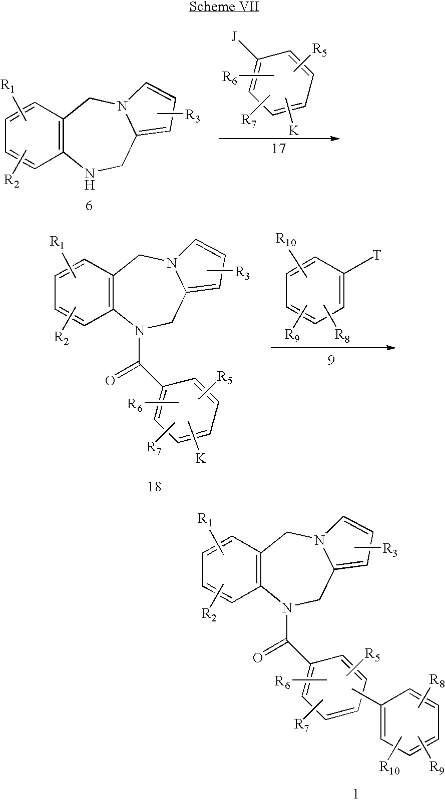
Thus, a tricyclic diazepine of formula (6) is treated with an appropriately substituted acylating agent such as, but not limited to, a halo aroyl halide of formula (17), preferably where J is COCl or COBr, and K is I, or Br, wherein R₅, R₆ and R₇ are hereinbefore defined, using any of the procedures hereinbefore described, to provide the acylated intermediate of general formula (18) of Scheme VII.

Alternatively, the acylating species of formula (17) can be a mixed anhydride of the corresponding carboxylic acid. Treatment of said mixed anhydride of general formula (17) with a tricyclic diazepine of formula (6) according to the procedure described hereinbefore yields the intermediate acylated derivative (18).

The acylating intermediate of formula (17) is ultimately chosen on the basis of its compatibility with the R₅, R₆ and R₇ groups, and its reactivity with the tricyclic diazepine of formula (6).

A Stille coupling reaction of the compound of formula (18), where K is I with an appropriately substituted organotin reagent such as, but not limited to, a trialkyltin(IV) derivative of formula (9), where R₈, R₉ and R₁₀ are hereinbefore defined, in the presence of a catalyst such as, but not limited to, tetrakis(triphenylphosphine) palladium (0), in an aprotic organic solvent such as, but not limited to, toluene and N,N-dimethylformamide, at temperatures ranging from about ambient to about 150° C. (cf. Farina et al., J. Org. Chem, 59, 5905 (1994) and references cited therein, affords the desired compounds of formula (1) wherein R₁, R₂, R₃, R₅, R₆, R₇, R₈, R₉ and R₁₀ are as defined hereinbefore. Preferably the trialkyltin(IV) derivative of formula (9) is a tri-n-butyltin(IV) derivative T is SnBu₃).

Alternatively, reaction of a compound of formula (18), where K is Cl, Br or I with an appropriately substituted aryl boronic acid of formula (9), where T is B(OH)₂ wherein R₅, R₆, R₇, R₈, R₉ and R₁₀ are hereinbefore defined, in a mixture of solvents such as, but not limited to, toluene-ethanol-water, and in the presence of a Pd(0) catalyst and a base such as, but not limited to, sodium carbonate, at temperatures ranging from ambient to the reflux temperature of the solvent, yields the desired compounds of formula (1) wherein R₁, R₂, R₃, R₅, R₆, R₇, R₈, R₉ and R₁₀ are as defined hereinbefore.

The preferred substituted aroyl chlorides or bromides of formula (17) of Scheme VII, where K is I, or Br and J is COCl or COBr, wherein R₅, R₆ and R₇ are as defined hereinbefore, are either available commercially, or are known in the art, or can be readily prepared by procedures analogous to those in the literature for the known compounds.

The intermediates of formula (9), where T is Sn(alkyl)₃, and particularly where alkyl is n-butyl, of Scheme VII are either commercially available, or can be conveniently prepared as shown in Scheme VIII from the corresponding bromo starting materials of formula (19) wherein R₈, R₉, and R₁₀ are hereinbefore defined, by first reacting them with n-butyl lithium followed by reaction of the intermediate lithiated species with a trialkyl tin(IV) chloride, such as, but not limited to, trimethyl tin(IV) chloride or tri-n-butyl tin(IV) chloride.

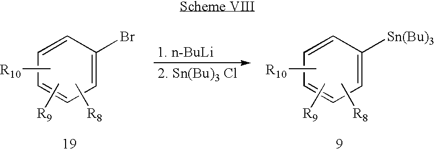

The preferred substituted aryl boronic acids of formula (9), where T is B(OH)₂ are either available commercially, or are known in the art, or can be readily prepared by procedures analogous to those in the literature for the known compounds.

The desired compounds of formula (1) of Scheme VII wherein B is B₁ and B₁ is 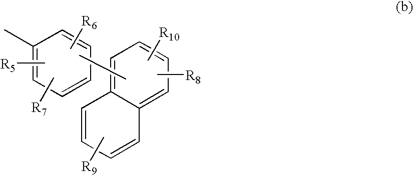
can be prepared in analogous fashion by replacing intermediates of formula (9) with appropriately substituted naphthyl intermediates.

Alternatively, as shown in Scheme IX, the appropriately substituted aroyl halides, preferably aroyl chlorides of formula (20, J=COCl) where R₅, R₆ and R₇ are hereinbefore defined, are reacted with a tricyclic diazepine of formula (6) to provide the intermediate bromides of formula (21). Subsequent reaction of (21) with an hexa alkyl-di-tin (preferably hexa-n-butyl-di-tin(IV)) in the presence of a Pd(0) catalyst such as tetrakis(tri-phenylphosphine)palladium(0) and lithium chloride or copper(I) salts, provides the stannane intermediate of formula (22). Further reaction of the tri-n-butyl tin(IV) derivative (22) with the appropriately substituted aryl halide of formula (23, M=bromo or iodo) wherein R₈, R₉, and R₁₀ are hereinbefore defined, in the presence of a Pd(0) catalyst such as tetrakis(triphenylphosphine) palladium(0), yields the desired compounds of formula (1)
wherein B is B₁ which is 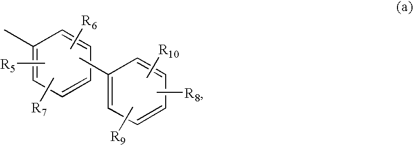
and R₁, R₂, R₃, R₅, R₆, R₇, R₈, R₉ and R₁₀ are defined hereinbefore.

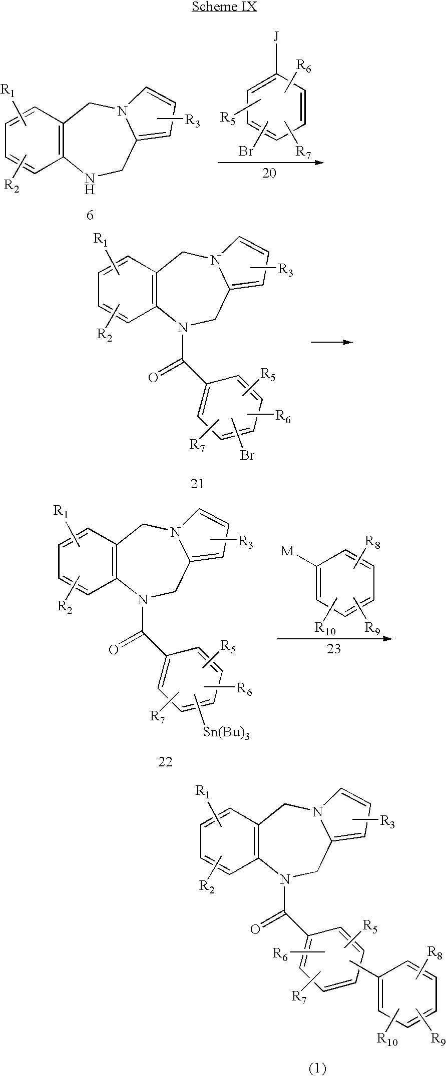

The desired compounds of formula (1) of Scheme IX wherein B is B₁ and B₁ is 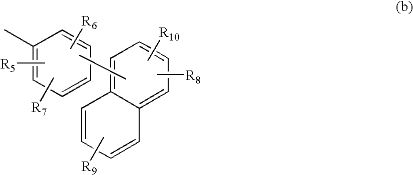
can be prepared in analogous fashion by replacing intermediates of formula (23) with appropriately substituted naphthyl intermediates.

Alternatively, the desired compounds of formula (1) of Scheme IX wherein B is B₁ and B₁ is 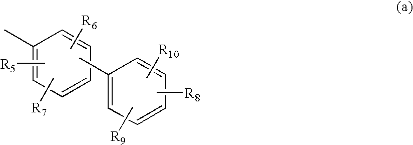
can be prepared as shown in Scheme X.

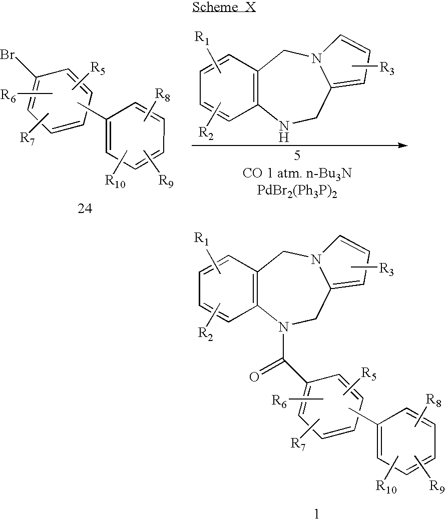

Thus, an appropriately substituted biphenyl of formula (24) wherein R₅, R₆, and R₇ are defined hereinbefore, is treated with carbon monoxide in the presence of a tricyclic diazepine of formula (6), a palladium(0) catalyst preferably PdBr₂(Ph₃P)₂ and a tertiary amine preferably n-tributylamine, in a solvent such as, but not limited to, anisole or dioxane, at temperatures ranging from about ambient to the reflux temperature of the solvent (cf. Schoenberg et al. J. Org. Chem. 39, 3327 (1974)) to provide the desired compounds of formula (1) wherein R₁, R₂, R₃, R₅, R₆, R₇, R₈, R₉ and R₁₀ are defined hereinbefore.

In analogous fashion one can prepare compounds of formula (1) of Scheme X wherein B is B₁ and B₁ is 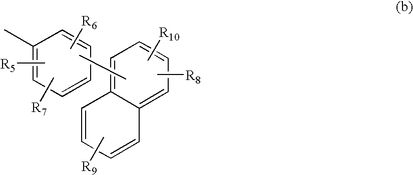
provided that the intermediates of formula (24) are replaced by the appropriately substituted naphthyl intermediates.

A preferred process for the preparation of the desired compounds of general formula (I), and corresponding formulas (II) and (III) of Schemes I-III wherein B is B₁ or B₂ wherein B₁ is selected from the group 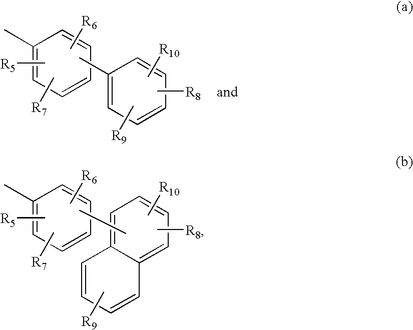
and B₂ is defined hereinbefore, is shown in Scheme XI

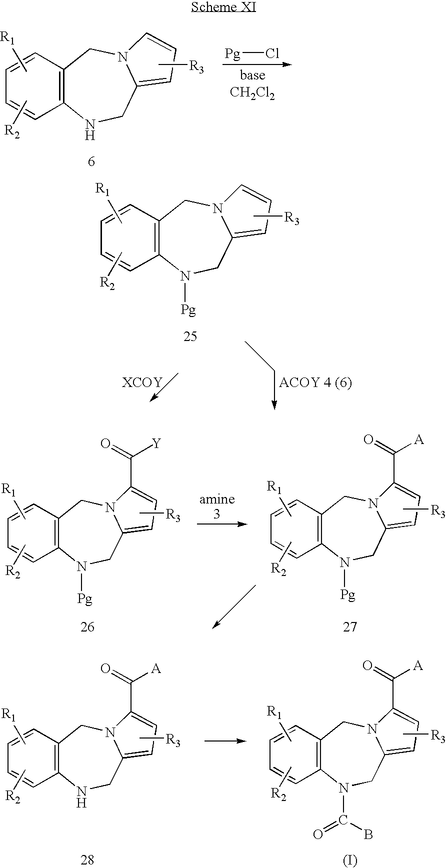

Thus, a tricyclic diazepine of formula (25) wherein R₁, R₂ and R₃ are defined hereinbefore, carrying a protecting group (Pg) such as, but not limited to, fluorenylalkoxycarbonyl group, preferably a fluorenylmethyloxycarbonyl group (Pg is Fmoc), or an alkoxycarbonyl protecting group preferably a tert-butyloxycarbonyl group (Pg is Boc) is reacted with an acid chloride under the conditions of Scheme I to provide the desired intermediate of formula (26). Subsequent reaction with an appropriate amine of formula (3) under the conditions of Scheme I provides the intermediate of formula (27) wherein A is A₁ as defined hereinbefore. Where the amine of formula (3) is an appropriately substituted pyridylamine or dialkylamine. Alternatively, treatment of (25) with an acid chloride of formula (4) under the conditions of Schemes II-III also yields the intermediate of formula (27) wherein A is A₂ as defined hereinbefore. The compound of formula (27) is then deprotected to yield the intermediate of formula (28) and, then acylated to the desired product of formula (I). Alternatively, the conversion of intermediate of formula (26) to the intermediate of formula (28) can be carried out in a single step by choosing appropriate reaction conditions.

Preferred processes for the preparation of compounds of formula (II) of Scheme I wherein B is B₁ and B₁ is 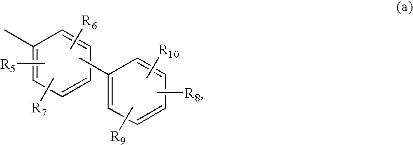
and R₁, R₂, R₃, R₅, R₆, R₇, R₈, R₉, and R₁₀are defined hereinbefore, also utilize acylation of the intermediate of formula (28) of Scheme XI with an acylating agent of formula (17) of Scheme VII, as shown in Scheme XII. Subsequent coupling of the intermediate of formula (29), where K is Br or I, with an appropriately substituted aryl boronic acid of formula (9), where T is B(OH)₂ in a mixture of solvents such as, but not limited to, dimethoxyethane and water or acetonitrile and water, in the presence of a Pd(0) catalyst such as, but not limited to, tetrakis(triphenylphosphine)palladium(0) or a Pd(II) catalyst such as, but not limited to, [1.1′-bis(diphenylphosphino)ferrocene]dichloro palladium(II), and a base such as, but not limited to, potassium or sodium carbonate, at temperatures ranging from about ambient to reflux, yields the desired compound of formula (II).

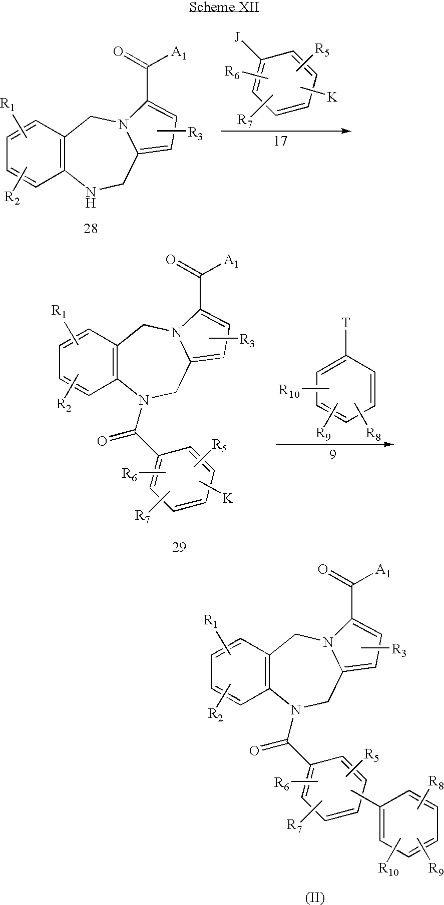

Alternatively, the preferred compounds of formula (II) of Scheme I wherein B is B₁ and B₁ is 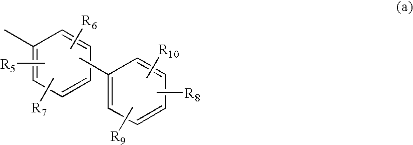
and R₁, R₂, R₃, R₅, R₆, R₇, R₈, R₉, and R₁₀ are defined hereinbefore, can be prepared as shown in Scheme XIII by acylation of the intermediate of formula (28) of Scheme XI with an acylating agent of formula (20) of Scheme IX.

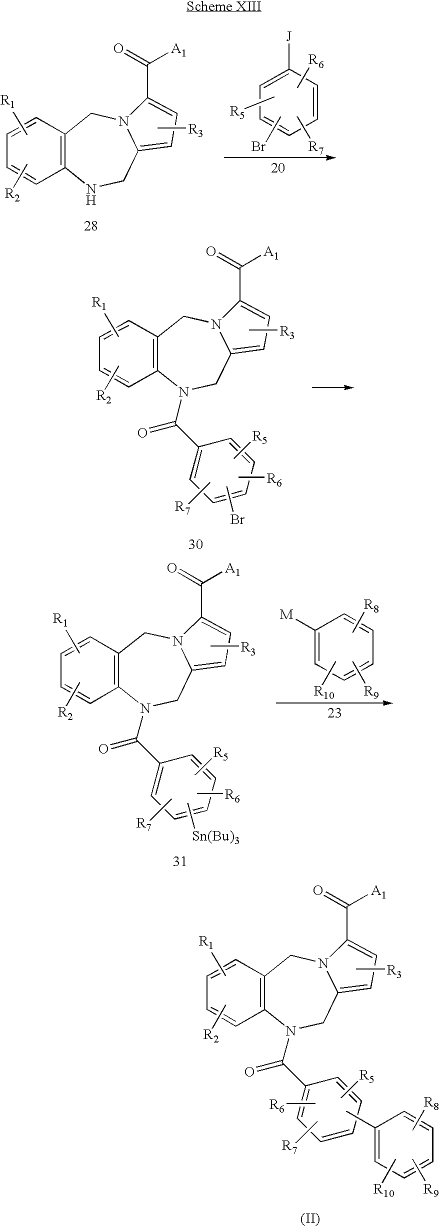

Alternatively, the preferred compounds of formula (II) of Scheme (I) wherein B is B₁ and B₁ is 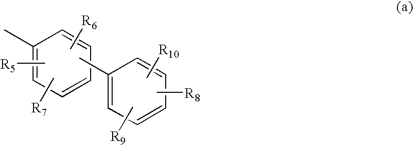
and R₁, R₂, R₃, R₅, R₆, R₇, R₈, R₉, and R₁₀ are defined hereinbefore, can be prepared by acylation of the intermediate of formula (28) of Scheme XI with an acylating agent of formula (7) of Scheme V, wherein J is hereinbefore defined, as shown in Scheme XIV

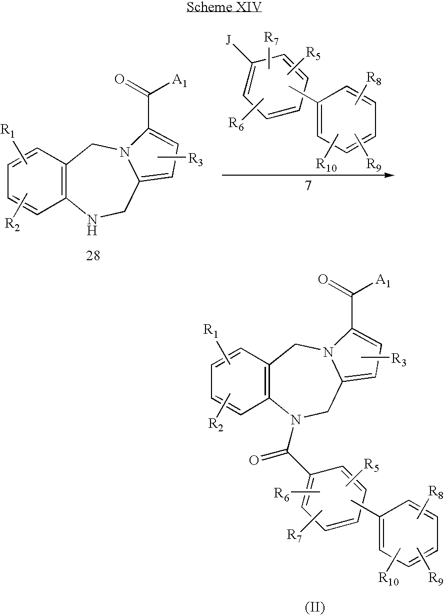

The tricyclic diazepines of formula (5) of Scheme III wherein B₂ is defined hereinbefore, can be conveniently prepared as shown in Scheme XV by reacting the diazepine of formula (6) with an appropriately substituted acylating agent such as, but not limited to, an aryloxy acetyl chloride or an aryloxy acetyl bromide of formula (32), where J is COCl or COBr, under the conditions of Scheme IV.

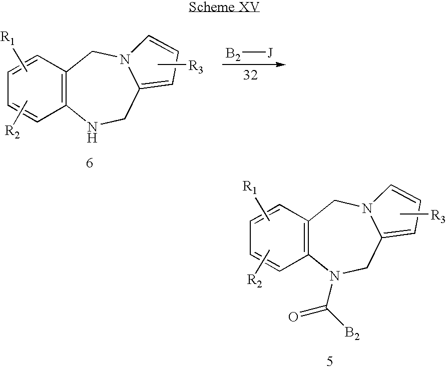

### Brief Description of Biological Test Procedure(s) and Text Summary of Results.

Pharmacology

The FSH antagonist activities of the compounds of this invention were demonstrated by evaluating representative compounds of this invention in the following test procedures.

### Follicle-Stimulating Hormone Receptor-Dependent CRE-Luciferase Reporter Gene Assay for the Identification of Follicle-Stimulating Hormone (FSH) Antagonists

This procedure was used to identify and determine the relative potencies of human FSH receptor antagonists using a Chinese hamster ovarian cell line that stably produces the human FSH receptor and a luciferase reporter gene regulated by cAMP response elements.

Materials and Methods: Reagents

Compound Vehicle: Stock compounds were solubilized in an appropriate vehicle, preferably phosphate buffered saline (PBS) or dimethyl sulfoxide (DMSO), at 30 mM. The compounds were subsequently diluted in DMSO to working dilutions of 1 and 20 or 30 mM for 2-dose testing format and 1 μM-10 mM for dose-response format. The DMSO dilutions were diluted 500-fold in sterile growth medium [D-MEM/F-12 (GIBCO/BRL; Grand Island N.Y.) containing 15 mM HEPES, 2 mM 1-glutamine, pyridoxine hydrochloride, phenol red and 5% FetalClone II (HyClone Laboratories, Inc; Logan, Utah), 0.2% DMSO, 100 units penicillin G/ml, and 100 μg streptomycin sulfate/ml (GIBCO/BRL)]. The concentration of the vehicle in each of the compound dilutions was the same.
Positive Controls: Purified human FSH (>98%) was purchased from Cortex Biochem, Inc. (San Leandro, Calif.) and WAY-162002 (an FSH-R thiazolidinone antagonist) was obtained from the Wyeth Research compound repository.
   Preparation of Cells

The CHO FSH-R 6CRE-Luc cells (1D7 cells) were obtained from Affymax (Palo Alto, Calif.). These Chinese hamster ovary cells (CHO—K1) were genetically engineered to stably express the recombinant human FSH receptor gene and a luciferase reporter gene under the regulation of 6 copies of a cAMP response element. The cells were plated one day prior to treatment into 96-well white opaque plates at a density of 50,000 cells/100 μl/well in growth medium. On the day of treatment, the growth medium was removed from the wells by aspiration and 50 μl of fresh growth medium was added to each well. The cells were incubated at 37° C. in a humidified incubator with 5% CO₂/95% air.

Assay

Test compounds diluted to 2×final concentration in growth medium containing 2×EC50 purified human FSH (0.8 ng/ml) were added to the wells to achieve a final volume of 100 μl of medium containing 0.25% (v/v) vehicle. The treated cells were incubated for 4 hours at 37° C. in a humidified incubator with 5% CO₂/95% air. At the end of-the incubation period, luciferase activity was measured by chemiluminescence using a commercially available kit (LucScreen, Tropix, Inc., Bedford, Mass.) according to the manufacturer's specifications, except that Buffer 1 and Buffer 2 were mixed together in equal proportion prior to the addition of 100 μl of the combined reagents to each well. Chemiluminescence was detected using a luminometer (EG & G Berthold Microlumat LB 96 P, Wallac, Gaithersburg, Md.) with chemiluminescence measured for 1 sec/well. Background luminescence was measured for each well prior to the addition of the LucScreen reagent.

Experimental Groups

In the 96-well 2-dose format, each compound was tested in duplicate at each dose. The controls were also tested in duplicate on each plate and consisted of vehicle control and 3 positive controls (EC₅₀ of phFSH (0.4 ng/ml), EC₁₀₀ of phFSH (1000 ng/ml), and IC₅₀ of 3-[(2S*,5R*)-5-{[2-(1H-Indol-3-yl)-ethylcarbamoyl]-methyl}-4-oxo-2-(5-phenylethynyl-thiophen-2-yl)-thiazolidin-3-yl]-benzamide (2 μM) in the presence of EC₅₀ of purified human FSH). One plate was used to test a maximum of 22 compounds.

In the 96-well dose-response format, each compound was tested in triplicate at each of 6 doses in the presence of the EC₅₀ of purified human FSH. The EC₅₀ of purified human FSH alone was tested in triplicate with each test compound. The doses chosen to test each compound were extrapolated from the initial 2-dose screening process. Along with the test compounds, purified human FSH was also tested in a dose response (0.03, 0.1, 0.3, 1, 3, 10, and 30 ng/ml) for a positive control and quality control. One plate was used for 3 test compounds and the FSH positive control.

Analysis of the Results

Luciferase activity is expressed as relative light units/sec/well. Luciferase activity in antagonist was compared to the appropriate negative and positive controls. For 2-dose testing, results are reported as luciferase activity and are expressed as % inhibition of the response obtained from the EC₅₀ of FSH. For dose-response testing, results are reported as IC₅₀ values. Data were analyzed statistically by one-way analysis of variance with appropriate weighting and transformation and relevant paired test as determined by Biometrics (Wyeth Research, Princeton, N.J.). IC₅₀ values were calculated using the Stat/Excel program developed by Biometrics with appropriate weighting and transformation.

Reference Compounds

Test compounds were compared to the effect of purified human FSH and 3-[(2S*,5R*)-5-{[2-(1H-Indol-3-yl)-ethylcarbamoyl]-methyl}-4-oxo-2-(5-phenylethynyl-thiophen-2-yl)-thiazolidin-3-yl]-benzamide in 2-dose format and EC₅₀ concentration of purified human FSH in dose-response format.

### REFERENCES

1. Kelton, C. A., Cheng, S. V. Y., Nugent, N. P., Schweickhardt, R. L., Rosenthal, J. L., Overton, S. A., Wands, G. D., Kuzeja, J. B., Luchette, C. A., and Chappel, S. C. (1992). The cloning of the human follicle stimulating hormone receptor and its expression in COS-7, CHO, and Y-1 cells. Mol. Cell. Endocrinol. 89:141-151.
2. Tilly, J. L., Aihara, T., Nishimori, K., Jia, X.-C., Billig, H., Kowalski, K. I., Perlas, E. A., and Hsueh, A. J. W. (1992). Expression of recombinant human follicle-stimulating hormone receptor: Species-specific ligand binding, signal transduction, and identification of multiple ovarian messenger ribonucleic acid transcripts. Endocrinology 131:799-806.
3. George, S. E., Bungay, P. J., and Naylor, L. H. (1997). Evaluation of a CRE-directed luciferase reporter gene assay as an alternative to measuring cAMP accumulation. J. Biomol. Screening 2:235-240.

### In vitro Bio-Assay of Agonists and Antagonists to the FSH Repeptor. Selectivity and Dependency of Agonists and Antagonists to the FSH Receptor

This assay was used to verify in vitro potency, efficacy, selectivity and receptor dependency of hits found to inhibit an FSH-R-CRE-luciferase driven reporter.

Methods: Reagents

Compound Vehicle: Stock compounds were solubilized in 100% DMSO (Sigma Chemical Co.) at a concentration of 30 mM. The compounds were subsequently diluted in sterile assay medium consisting of Opti-MEM® I (Life Technologies) with 0.1% (w/v) BSA (Sigma), prior to use in the bio-assay. The final concentration of DMSO in the assay is 0.1%.

Preparation of CHO-3D2 Cells

The day prior to the experiment, CHO-3D2 cells (hFSH-R)(1) were plated into 96-well tissue culture plates (Falcon) at a density of 30,000 cells/well in DMEM/F12 medium (Life Technologies) supplemented with 5% Fetal Clone II (Hyclone), 2 mM L-glutamine (Life Technologies) and penicillin/streptomycin (100 U/ml, Life Technologies). Plated cells are then incubated at 37° C. in a humidified 5% CO₂/95% air, atmosphere.

Assay:

On the day of the assay, cells were washed three times with 100 μl/well of assay medium consisting of Opti-MEM® I (Life Technologies) with 0.1% (w/v) BSA (Sigma). Medium was removed and 100 μl of assay medium was added to each well. Plates were incubated for an additional 30 minutes at 37° C. Medium was then removed and cells were challenged for 30 minutes at 37° C. in 50 μl of assay media containing vehicle, purified hFSH (>95% pure; Cortex Biochem, Inc., San Leandro, Calif.) in the presence or absence of test compounds. Reactions were terminated by the addition of 50 μl of 0.2N hydrochloric acid to each well and cAMP-accumulation was measured by radioimmunoassay (RIA) using a commercially available kit (Amersham).

Experimental Groups

All test compounds were evaluated in a dose-response paradigm ranging from 0.01 to 30 μM. Controls and test compounds were evaluated in quadruplicate in a 96-well format. Cells were treated with vehicle, hFSH at EC₂₀ (1.85 ng/mL is 53 pM), or the compounds in the presence or absence of hFSH at its EC₂₀ dose. The ability of the compounds to inhibit the cAMP-accumulation induced by hFSH was evaluated by RIA.

In every assay the EC₂₀ concentration was calculated and only those experiments in which the EC₂₀ concentrations were equal to 1.85±0.4 ng/mL were accepted as valid. In the 96-well format, the first column contained the negative control (assay media+0.1% DMSO), the second column contained the positive control, hFSH at its EC₂₀+0.1% DMSO (1.85 ng/ml or 53 pM), followed by six concentrations of the compound ranging from 0.03-30 μM in the presence of the hFSH at its EC₂₀ concentration (1.85 ng/ml or 53 pM).

Along with the test compounds, FSH was also run as a positive control in the agonist mode using concentrations ranging from 0.1-1000 ng/ml.

Selectivity Studies

cAMP accumulation assays using CHO-25 (hTSH-R) cells were performed as described above for the CHO-3D2 cells with the following exceptions: CHO-25 cells were plated at a density of 50,000 cells/well (2). All test compounds were evaluated in a dose-response paradigm ranging from 0.01 to 30 μM. Controls and test compounds were evaluated in quadruplicate. Cells were treated with vehicle, hTSH at EC₂₀ (5nM; hTSH>98% pure, Cortex Biochem, Inc.), or the compounds in the presence or absence of the hTSH at its EC₂₀ concentration. The ability of the compounds to inhibit cAMP-accumulation induced by hTSH was evaluated by RIA.

Along with the test compounds, hTSH was also run as a positive control in the agonist mode using concentrations ranging from 0.01 μM-1000 l μM.

Non-Receptor Mediated Responses:

cAMP-accumulation assays using CHO-K1 (parental cell line) cells were performed as described above for the CHO-3D2 cells. All test compounds were evaluated in a dose-response paradigm ranging from 0.01 to 30 μM. Controls and test compounds were evaluated in quadruplicate. Cells were treated with vehicle, 5 μM forskolin that induces the equivalent fmol/ml concentration of cAMP-accumulation induced by the hFSH at its EC₂₀ (5 μM forskolin, Sigma Chemical Co; previously calculated during characterization of the bio-assays), or the compounds in the presence or absence of the 5 μM forskolin. The ability of the compounds to inhibit the cAMP-accumulation induced by forskolin was evaluated by RIA.

Along with the test compounds, forskolin was also run as a positive control in agonist mode using concentrations ranging from 0.01 μM to 1000 μM.

Analysis of Results

cAMP accumulation is expressed as fmol/ml. cAMP accumulation in the agonist mode, or the ability of the compound to inhibit hFSH-, hTSH-, or forskolin-induced cAMP-accumulation in the antagonist mode, was compared to the appropriate negative and positive controls. Data were analyzed by one-way analysis of variance and significant differences between treatments and control determined by Least Significant Difference test.

Reference Compounds

Test compounds were compared to the effect of purified human FSH. In the paradigm, hFSH induced a concentration-dependent increase in cAMP accumulation, with apparent EC₈₀=22.55 ng/ml, EC₅₀=6.03 ng/ml and EC₂₀=1.85 ng/ml, calculated using a four-parameter logistic equation. The same comparison was performed with hTSH and forskolin.

Biological Activity

Based on the results obtained in the standard pharmacological test procedures, the compounds of this invention were shown to block cellular function of FSH, in vitro, including the production of second messenger cAMP and estradiol in rat ovarian granulosa cells. Representative compounds of this invention were found to selectively interact with the FSH receptor, but do not antagonize binding of FSH to its receptor (Table 1).

As such, the compounds of this invention may be useful as female contraceptive agents.

TABLE 1

CRE

| | |
|---|---|
| % | cAMP |

| | inhibition | IC50 | IC50 | % |
|---|---|---|---|---|
| Example | (μM) | .μM) | .μM) | efficacy |
| 1 | 4(30) | | | |
| 2 | | 10.66 | | |
| 3 | | 6.82 | 2 | 78 |
| 4 | 22(30) | | | |
| 5 | | >30 | | |
| 6 | | >30 | | |
| 7 | | >30 | | |
| 8 | | >30 | | |
| 9 | | >30 | | |
| 10 | | >30 | | |
| 11 | 8(30) | | | |
| 12 | | >30 | | |
| 13 | 14(30) | | | |
| 14 | | >30 | | |
| 15 | | 12.11 | | |

### EXAMPLES

### Example 1

### 1-{10-[(2,2′-Dimethyl-1,1′-biphenyl-4-yl)carbonyl]-10,11-dihydro-5H-pyrrolo[2,1-c][1,4]benzodiazepin-3-yl}-2-(pyridin-3-ylamino)ethanone formic acid salt

### Step A. (10,11-Dihydro-5H-pyrrolo[2,1-c][1,4]benzodiazepin-10-yl)-(2,2′-dimethyl-biphenyl-4-yl)-methanone

A solution of 0.45 g (0.002 mole) of 2,2′-dimethyl-1,1′-biphenyl-4-carboxylic acid in 50 mL of thionyl chloride was heated under reflux overnight. The excess thionyl chloride was stripped off in vacuo. To the residue was added 0.37 g (0.002 mole) of 10,11-dihydro-5H-pyrrolo[2,1-c][1,4]benzodiazepine and 50 mL of 1,4-dioxane followed by 0.24 g (0.002 mole) of N,N-dimethylaniline. After standing for three hours, the reaction solution was poured into 300 mL of water to provide 0.6 g of title compound which was used directly in the next step after drying.

MS [(+)ESI, m/z]: 393 [M+H]⁺.

### Step B. 2-Chloro-1-[10-(2,2′-dimethyl-biphenyl-4-carbonyl)-10,11-dihydro-5H-pyrrolo[2,1-c][1,4]benzodiazepine-3-yl]-ethanone

A solution containing 0.992 g (0.001 mole) of (10,11-dihydro-5H-pyrrolo[2,1-c][1,4]benzodiazepin-10-yl)-(2,2′-dimethyl-1,1′-biphenyl-4-yl)-methanone of Step A and 0.16 g (0.001 mole) of chloroacetyl chloride in 20 mL of 1,4-dioxane was heated under reflux with stirring for two hours. The solvent was removed in vacuo and the residue was used directly in the next step.

MS [(+)ESI, m/z]: 469 [M+H)⁺

### Step C. 1-{10-[(2,2′-Dimethyl-1,1′-biphenyl-4-yl)carbonyl]-10,11-dihydro-5H-pyrrolo[2,1-c][1,4]benzodiazepin-3-yl}-2-(pyridin-3-ylamino)ethanone formic acid salt

To the crude 2-chloro-1-[10-(2,2′-dimethyl-1,1′-biphenyl-4-carbonyl)-10,11-dihydro-5H-pyrrolo[2,1-c][1,4]benzodiazepine-3-yl]-ethanone of Step B was added 0.94 g (0.010 mole) of 3-aminopyridine. The reaction mixture was heated neat to the melting temperature and kept at this temperature for twenty minutes. It was then allowed to cool to room temperature and the residue was washed several times with water to remove the excess 3-aminopyridine. The remaining crude product was purified by hplc (formic acid/acetonitrile/water) to provide the title compound as the formic acid salt.

MS [(+)ESI, m/z]: 527 [M+H]⁺.

### Example 2

### 1-[10-(1,1′-Biphenyl-4-ylcarbonyl)-10,11-dihydro-5H-pyrrolo[2,1-c][1,4]benzodiazepin-3-yl]-3-pyridin-3-ylpropan-1-one formic acid salt

A mixture of 1.13 g (0.003 mole) of (5H,10)-[(1.1′-biphenyl-4-yl)carbonyl]-10,11-dihydro-5H-pyrrolo[2,1-c][1,4]benzodiazepine and 0.003 mole of 3-pyridin-3-yl-propionyl chloride hydrochloride (generated via the reaction of 3-pyridinyl-3-yl-propionic acid with thionyl chloride) was heated to the melting point, keeping the temperature at this level for twenty minutes. The reaction mixture was allowed to cool to room temperature and the residue was neutralized with 10% aqueous sodium bicarbonate and then washed with water. The crude product thus obtained was purified by HPLC (formic acid/acetonitrile/water) to provide the title compound as the formic acid salt.

MS [(+)ESI, m/z]: 498 [M+H]⁺

### Example 3

### 1-{10-[(2′-Methoxy-1,1′-biphenyl-4-yl)carbonyl]-10,11-dihydro-5H-pyrrolo[2,1-c][1,4]benzodiazepin-3-yl}-3-pyridin-3-ylpropan-1-one

A mixture of (2′-methoxy-1,1′-biphenyl-4-yl)-(5H,11H-pyrrolo[2,1-c][1,4]benzodiazepin-10-yl)-methanone (0.503 g, 1.27 mmole), 3-pyridin-3-yl-propionyl chloride hydrochloride salt (0.473 g, 2.3 mmole), 2,6-lutidine (0.478 g, 4.46 mmole) and N-methyl-2-pyrrolidinone (1.5 mL) was heated under nitrogen at 120° C. for 30 minutes. The mixture was diluted with 30 mL of dichloromethane. The organic phase was washed with 1 N sodium hydroxide and brine, and dried over anhydrous magnesium sulfate. The solvent was removed in vacuo and the residue was purified by preparative HPLC, Primesphere 10 C18 5×25 cm column, 48% acetonitrile in water containing 0.1% trifluoroacetic acid, 100 mL/min, 254 nm detection. The eluate was neutralized with aqueous sodium hydroxide and the volatiles removed in vacuo. The residue was extracted with dichloromethane, the extracts were dried over anhydrous magnesium sulfate and evaporated to provide the title compound as an off-white amorphous solid.

MS [(+)ESI, m/z]: 528.18 [M+H]⁺

### Example 4

### {10-[(4-Chloro-2-methylphenoxy)acetyl]-10,11-dihydro-5H-pyrrolo[2,1-c][1,4]benzodiazepin-3-yl}(4-chlorophenyl)methanone

### Step A. 4-Chloro-o-tolyloxyacetic acid chloride

To a cold suspension of 4-chloro-o-tolyloxyacetic acid (17.4 mmol) in 40 mL of dry dichloromethane was added oxalyl chloride (39.15 mmol) followed by one drop of N,N-dimethylformamide. Bubbling began immediately. After 30 minutes the reaction mixture was warmed in a 45° oil bath for 1.5 h. The solution was cooled to room temperature and all volatiles were removed by evaporation. Move dry dichloromethane was added and this was again evaporated in vacuo. Finally, dry toluene was added to the residue and this was evaporated at reduced pressure. The crude acid chloride was used without further purification in the following step.

### Step B. 10-[(4-Chloro-2-methylphenoxy)acetyl]-10,11-dihydro-5H-pyrrolo[2,1c][1,4]benzodiazepine

To a solution of the crude acid chloride of Step A (17.4 mmol) in dichloromethane (25 mL) was added a solution of 10,11-dihydro-5H-pyrrolo[2,1-c][1,4]benzodiazepine (17.4 mmol) and triethylamine (19.14 mmol) in dichloromethane (25 mL) in a rapid dropwise fashion. After stirring for one hour at room temperature, the reaction mixture was washed with 0.1 N aqueous hydrochloric acid (2×) and water (1×), dried over anhydrous sodium sulfate, and evaporated. The product was isolated by crystallization from hot ethyl acetate/tert-butyl methyl ether (2/1), mp 166-167° C.

MS [(+)ESI, m/z]: 367 [M+H]⁺

Anal. Calcd for C₂₁H₁₉ClN₂O₂: C, 68.76; H, 5.22; N, 7.64. Found: C, 68.53; H, 5.18; N, 7.53.

### Step C. {10-[(4-Chloro-2-methylphenoxy)acetyl}-10,11-dihydro-5H-pyrrolo[2,1-c][1,4]benzodiazepin-3-yl}(4-chlorophenyl)methanone

A solution of 10-[(4-chloro-2-methylphenoxy)acetyl]-10,11-dihydro-5H-pyrrolo[2,1-c][1,4]benzodiazepine of Step B (0.68 mmol), 4-chlorobenzoyl chloride (1.02 mmol) and 2,6-lutidine (1.02 mmol) in N-methyl-2-pyrrolidinone (0.33 mL) was heated to 115° C. under a nitrogen atmosphere for 16 hours. To the cooled reaction mixture was added dichloromethane (5 mL). The organic solution was washed with water (2×), 1N aqueous hydrochloric acid (1×), 0.5 N aqueous sodium hydroxide (1×), and water (1×). The organic phase was dried over anhydrous sodium sulfate, and evaporated. HPLC was used for the purification of the title compound which was then crystallized from hot ethyl acetate/hexane, mp 175-176° C.

MS [(+)ESI, m/z]: 505 [M+H]⁺

Anal. Calcd for C₂₈H₂₂Cl₂N₂O₃: C, 66.54; H, 4.39; N, 5.54. Found: C, 66.58; H, 4.60; N, 5.36

### Example 5

### 1-{10-[(4-Chloro-2-methylphenoxy)acetyl]-10,11-dihydro-5H-pyrrolo[2,1-c][1,4]benzodiazepin-3-yl}-3-phenylpropan-1-one

The title compound (m.p. 130-134° C.) was prepared from the 10-[(4-chloro-2-methylphenoxy)acetyl]-10,11 -dihydro-5H-pyrrolo[2,1-c][1,4]benzodiazepine of Example 4, Step B and phenyl propionyl chloride in the manner of Example 4, step C.

MS [(+)ESI, m/z]: 499 [M+H]⁺

Anal. Calcd for C₃₀H₂₇ClN₂O₃.0.15C₅H₁₀O₂: C, 71.75; H, 5.55; H, 5.47 Found: C, 71.77; H, 5.54; N, 5.46.

### Example 6

### {10-[(4-Chloro-2-methylphenoxy)acetyl]-10,11-dihydro-5H-pyrrolo[2,1-c][1,4]benzodiazepin-3-yl}(1-naphthyl)methanone

The title compound (m.p. 130-134° C.) was prepared from the 10-[(4-chloro-2-methylphenoxy)acetyl]-10,11-dihydro-5H-pyrrolo[2,1-c][1,4]benzodiazepine of Example 4, Step B and 1-naphthoyl chloride in the manner of Example 4, step C.

MS [(+)ESI, m/z]: 521 [M+H]⁺

Anal. Calcd for C₃₂H₂₅ClN₂O₃.1.2C₅H₁₀O₂: C, 70.52; H, 5.56; N, 4.47. Found: C, 70.39; H, 5.30; N, 4.60.

### Example 7

### 1,1′-Biphenyl-4-yl{10-[(4-chloro-2-methylphenoxy)acetyl]-10,11-dihydro-5H-pyrrolo[2,1-c][1,4]benzodiazepin-3-yl}methanone

The tile compound (m.p. 102-105° C.) was prepared from the 10-[(4-chloro-2-methylphenoxy)acetyl]-10,11-dihydro-5H-pyrrolo[2,1-c][1,4]benzodiazepine of Example 4, Step B and 4-(1,1′-biphenyl) carbonyl chloride in the manner of Example 4, step C.

MS [(+)ESI, m/z]: 547 [M+H]⁺

Anal. Calcd for C₃₄H₂₇ClN₂O₃.C₅H₁₀O₂: C, 73.43; H, 5.23; N, 4.81. Found: C, 73.34; H, 4.93; N, 4.90.

### Example 8

### (4-Tert-butylphenyl){10-[(4-chloro-2-methylphenoxy)acetyl]-10,11-dihydro-5H-pyrrolo[2,1-c][1,4]benzodiazepin-3-yl}methanone

The title compound (m.p. 168° C.) was prepared from 10-[(4-chloro-2-methylphenoxy)acetyl]-10,11-dihydro-5H-pyrrolo[2,1-c][1,4]benzodiazepine of Example 4, Step B and 4-tert-butyl benzoyl chloride in the manner of Example 4, step C.

MS [(+)ESI, m/z]: 527 [M+H]⁺

Anal. Calcd for C₃₂H₃₁ClN₂O₃: C, 72.92; H, 5.93; N, 5.31. Found: C, 72.53; H, 5.92; N, 5.20.

### Example 9

### 1,1′-Biphenyl-2-yl{10-[(4-chloro-2-methylphenoxy)acetyl]-10,11-dihydro-5H-pyrrolo[2,1-c][1,4]benzodiazepin-3-yl}methanone

The title compound was prepared from 10-[(4-chloro-2-methylphenoxy)acetyl]-10,11-dihydro-5H-pyrrolo[2,1-c][1,4]benzodiazepine of Example 4, Step B and 2-(1,1′-biphenyl) carbonyl chloride in the manner of Example 4, step C.

MS [(+)ESI, m/z]: 547.1 [M+H]⁺

### Example 10

### {10-[(4-Chlorophenoxy)acetyl]-10,11-dihydro-5H-pyrrolo[2,1-c][1,4]benzodiazepin-3-yl}(4-chlorophenyl)methanone

### Step A. 10-[(4-Chlorophenoxy)acetyl]-10,11-dihydro-5H-pyrrolo[2,1c][1,4]benzodiazepine

The title compound (mp 120-122° C.) was prepared from 10,11-dihydro-5H-pyrrolo[2,1-c][1,4]benzodiazepine and 4-chlorophenoxyacetyl chloride in the manner of Example 4, step B.

MS [(+)ESI, m/z]: 353 [M+H]⁺

Anal. Calcd for C₂₀H₁₇ClN₂O₂: C, 68.09; H, 4.86; N, 7.94. Found: C, 67.82; H, 4.87; N, 7.87.

### Step B. {10-[(4-Chlorophenoxy)acetyl]-10,11-dihydro-5H-pyrrolo[2,1c][1,4]benzodiazepin-3-yl}(4-chlorophenyl)methanone

The title compound (m.p. 195° C.) was prepared from 10-[(4-chlorophenoxy)acetyl]-10,11-dihydro-5H-pyrrolo[2,1-c][1,4]benzodiazepine of Example 14 and 4-chlorobenzoyl chloride in the manner of Example 4, step C.

MS [(+)ESI, m/z]: 491 [M+H]⁺

Anal. Calcd for C₂₇H₂₀Cl₂N₂O₃: C, 66.00; H, 4.10; N, 5.70. Found: C, 65.67; H, 4.07; N, 5.45.

### Example 11

### 1-{10-[(4-Chlorophenoxy)acetyl]-10,11-dihydro-5H-pyrrolo[2,1-c][1,4]benzodiazepin-3-yl}-3-phenylpropan-1-one

The title compound (m.p. 126-128° C.) was prepared from 10-[(4-chlorophenoxy)acetyl]-10,11-dihydro-5H-pyrrolo[2,1-c][1,4]benzodiazepine of Example 10, Step A and phenyl propionyl chloride in the manner of Example 4, step C.

MS [(+)ESI, m/z]: 485 [M+H]⁺

Anal. Calcd for C₂₉H₂₅ClN₂O₃: C, 71.82; H, 5.20; N, 5.78. Found: C, 71.52; H, 5.31; N, 5.66.

### Example 12

### (4-tert-Butylphenyl){10-[(4-chlorophenoxy)acetyl]-10,11-dihydro-5H-pyrrolo[2,1-c][1,4]benzodiazepin-3-yl}methanone

The title compound (m.p. 171° C.) was prepared from 10-[(4-chlorophenoxy)acetyl]-10,11-dihydro-5H-pyrrolo[2,1-c][1,4]benzodiazepine of Example 10, Step A and 4-tert-butyl benzoyl chloride in the manner of Example 4, step C.

MS [(+)ESI, m/z]: 513 [M+H]⁺

Anal. Calcd for C₃₁H₂₉ClN₂O₃.0.15C₅H₁₀O₂: C, 72.12; H, 5.78; N, 5.32. Found: C, 72.04; H, 5.51; N, 5.30.

### Example 13

### 1,1′-Biphenyl-4-yl{10-[(4-chlorophenoxy)acetyl]-10,11-dihydro-5H-pyrrolo[2,1-c][1,4]benzodiazepin-3-yl}methanone

The title compound (m.p. 155-157° C.) was prepared from 10-[(4-chlorophenoxy)acetyl]-10,11-dihydro-5H-pyrrolo[2,1-c][1,4]benzodiazepine of Example 10, Step A and 4-(1,1′-biphenyl) carbonyl chloride in the manner of Example 4, step C. MS [(+)ESI, m/z]: 533.1 [M+H]⁺

### Example 14

### 1,1′-Biphenyl-2-yl{10-[(4-chlorophenoxy)acetyl]-10,11-dihydro-5H-pyrrolo[2,1-c][1,4]benzodiazepin-3-yl}methanone

The title compound was prepared from 10-[(4-chlorophenoxy)acetyl]-10,11-dihydro-5H-pyrrolo[2,1-c][1,4]benzodiazepine of Example 10, step A and 2-(1,1′-biphenyl) carbonyl chloride in the manner of Example 4, step C.

MS [(+)ESI, m/z]: 533.1 [M+H]⁺

### Example 15

### 1-{10-[(2′-Methyl-1,1′-biphenyl-4-yl)carbonyl]-10,11-dihydro-5H-pyrrolo[2,1-c][1,4]benzodiazepin-3-yl}-3-pyridin-3-ylpropan-1-one

The title compound (m.p. 135-136° C.) was prepared from (2′-methyl-1,1′-biphenyl-4-yl)-(5H,11H-pyrrolo[2,1-c][1,4]benzodiazepin-10-yl)-methanone and 3-pyridin-3-yl-propionyl chloride in the manner of Example 4, step C.

MS [(+)ESI, m/z]: 512.18 [M+H]⁺

Anal. Calcd for C₃₄H₂₉N₃O₂.0.10C₅H₁₀O₂: C, 79.39; H, 5.77; N, 8.07. Found: C, 79.29; H, 5.88; N, 8.16.

All references, including but not limited to articles, texts, patents, patent applications, and books, cited herein are hereby incorporated by reference in their entirety.

What is claimed is:

1. A compound represented by the formula I 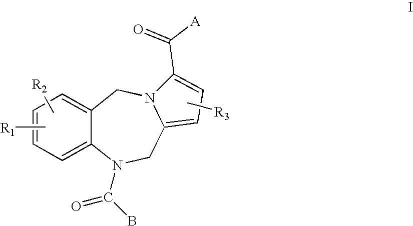
or a pharmaceutically acceptable salt thereof,
wherein
R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];
R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B is B₁ or B₂,
wherein B₁ is selected independently from the group consisting of 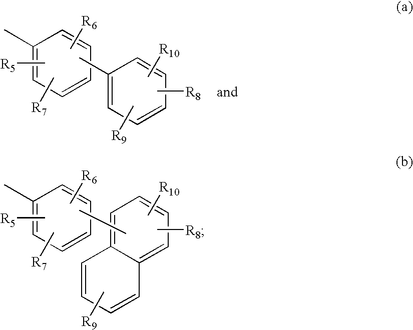
wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxyalkyl, hydroxy(C₁-C₆) alkyl, alkyloxyalkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈)cycloalkyloxycarbonyl, aryl(C₁-C₆)alkyloxycarbonyl, carbamoyl, —O—CH₂—CH═CH₂, (C₁-C₆)alkyl substituted with 1-3 halogen atoms, trihalomethyl, trifluoromethyl, halogen, OCF₃, thioalkyl, thio(C₁-C₆) alkyl, —C(O) alkyl, —C(O)aryl optionally substituted by alkyl; hydroxy, —CH(OH)alkyl, —CH(alkoxy)alkyl, nitro, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCONR₁₃R₁₄, —(CH₂)ₚCOOR₁₂, —CH═NOH, —CH═NO—(C₁-C₆) alkyl, trifluoromethylthio, 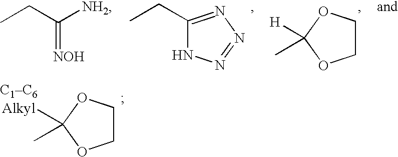
R₁₁ and R₁₂ are each independently hydrogen, alkyl, cycloalkyl, or C₃-C₈ cycloalkyl;
R₁₃ and R₁₄ are each independently hydrogen, alkyl, cycloalkyl, or C₃-C₈ cycloalkyl;
or R₁₃ and R₁₄ can be taken together with the nitrogen to which they are attached to form a 4-6 membered saturated ring optionally containing up to two atoms selected from O, S or N;
p is 0 or 1;
A is A₁ or A₂, wherein
A₁ is selected from 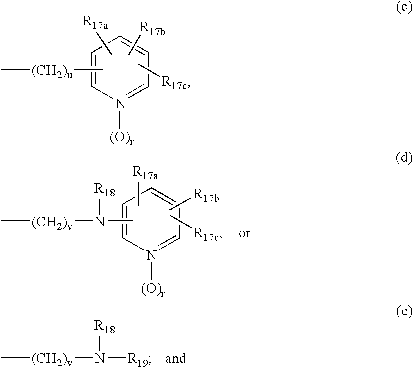
A₂ is selected from 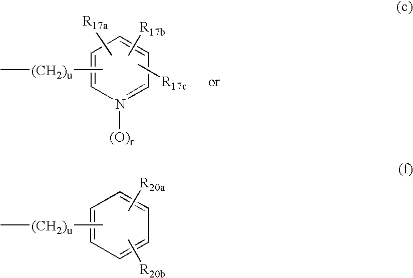
provided that when A is A₂, then B is B₂ wherein B₂ is 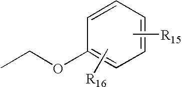
wherein R₁₅ and R₁₆ are selected independently from the group consisting of hydrogen, alkyl, and halogen;
wherein
R₁₇ₐ, R_{17b}, and R_{17c} are each independently selected from the group consisting of hydrogen, alkyl, halogen, hydroxy, aryloxy, and hydroxyalkyl;
u is the integer 0, 1, 2, 3, or 4;
v is the integer 1, 2, 3, or 4;
r is 0 or 1;
R₁₈ is hydrogen or alkyl; and
R₁₉ is a cycloalkylamine.
R₂₀ₐ and R_{20b} are each independently selected from the group consisting of hydrogen, alkyl, halogen, or aryl; or R₂₀ₐ and R_{20b} can be taken together with the aryl to which they are attached to form an aromatic bicycle having up to 10 total ring atoms.

2. A compound according to claim 1, wherein A is A₁.

3. A compound according to claim 2, wherein A₁ is

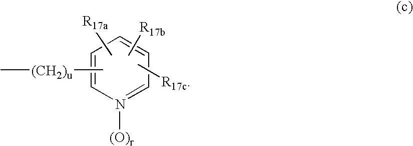

4. A compound according to claim 2, wherein A₁ is

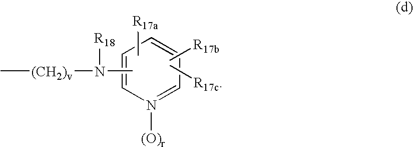

5. A compound according to claim 2, wherein A₁ is

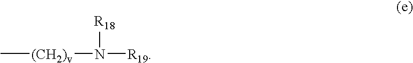

6. A compound according to claim 2, wherein B is B₁, and B₁ is

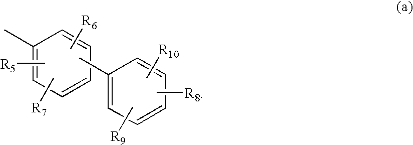

7. A compound according to claim 2, wherein B is B₁ and B₁ is

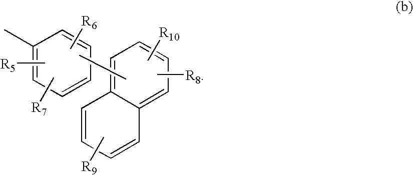

8. A compound according to claim 1, wherein A is A₂ and B is B₂.

9. A compound according to claim 8, wherein A₂ is

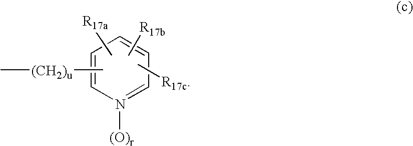

10. A compound according to claim 8, wherein A₂ is

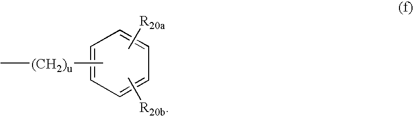

11. A compound represented by the formula II 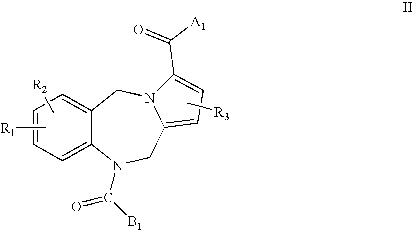
or a pharmaceutically acceptable salt thereof,
wherein
R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];
R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B₁ is selected independently from the group consisting of 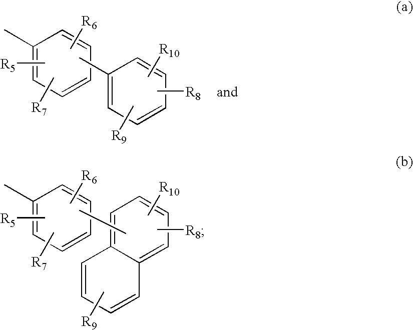
wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxyalkyl, hydroxy(C₁-C₆) alkyl, alkyloxyalkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, (C₁-C₆)alkyl substituted with 1-3 halogen atoms, trihalomethyl, trifluoromethyl, halogen, OCF₃, thioalkyl, thio(C₁-C₆) alkyl, —C(O) alkyl, —C(O)aryl optionally substituted by alkyl; hydroxy, —CH(OH)alkyl, —CH(alkoxy)alkyl, nitro, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCONR₁₃R₁₄, —(CH₂)ₚCOOR₁₂, —CH═NOH, —CH═NO—(C₁-C₆) alkyl, trifluoromethylthio, 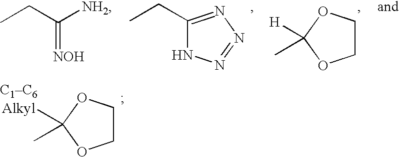
R₁₁ and R₁₂ are each independently hydrogen, alkyl, cycloalkyl, or C₃-C₈ cycloalkyl;
R₁₃ and R₁₄ are each independently hydrogen, alkyl, cycloalkyl, or C₃-C₈ cycloalkyl;
or R₁₃ and R₁₄ can be taken together with the nitrogen to which they are attached to form a 4-6 membered saturated ring optionally containing up to two atoms selected from O, S or N;
p is 0 or 1;
A₁ is selected from the group consisting of 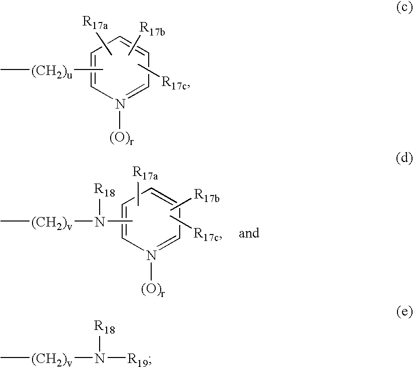
R₁₇ₐ, R_{17b}, and R_{17c} are each independently selected from the group consisting of hydrogen, alkyl, halogen, hydroxy, aryloxy, and hydroxyalkyl;
u is 0, 1, 2, 3, or 4;
v is 1, 2, 3, or 4;
r is 0 or 1;
R₁₈ is hydrogen or alkyl; and
R₁₉ is a cycloalkylamine.

12. A compound according to claim 11, wherein A₁ is

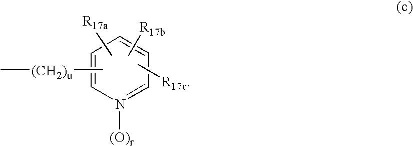

13. A compound according to claim 12, wherein u is 2.

14. A compound according to claim 12, wherein r is 0.

15. A compound according to claim 12, wherein A₁ is

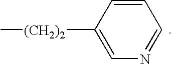

16. A compound according to claim 12, wherein B₁ is

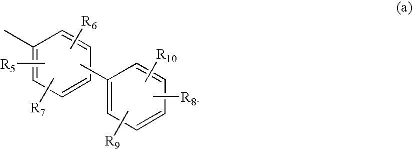

17. A compound according to claim 16, wherein B₁ is

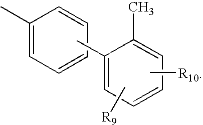

18. A compound according to claim 16 wherein B₁ is

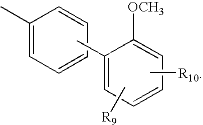

19. A compound according to claim 12 represented by the following formula:

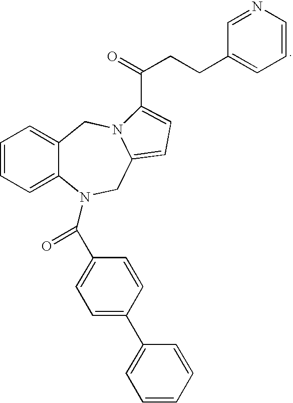

20. A compound according to claim 12 represented by the following formula:

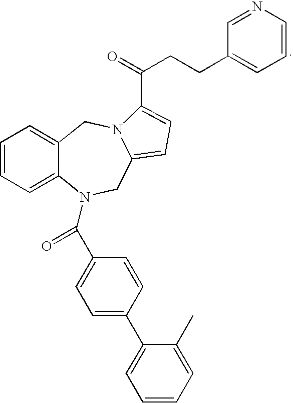

21. A compound according to claim 12 represented by the following formula:

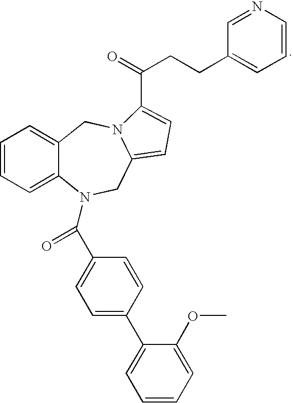

22. A compound according to claim 11, wherein A₁ is

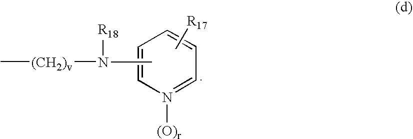

23. A compound according to clam 22, represented by the following formula:

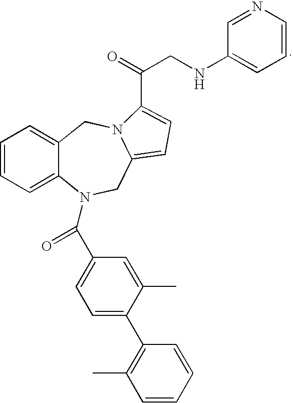

24. A compound according to claim 22, wherein B₁ is

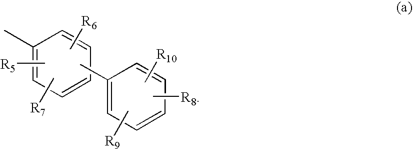

25. A compound according to claim 11, wherein A₁ is

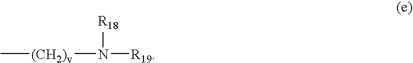

26. A compound represented by the formula III 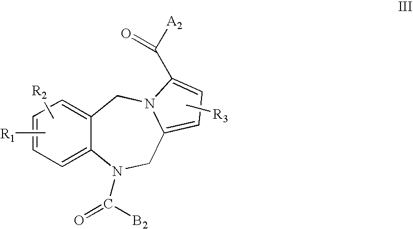
or a pharmaceutically acceptable salt thereof,
wherein
R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C_{-C}₆) alkylamino, and —NHCO[(C₁-C₆) alkyl];
R₃ is a substituent selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B₂is 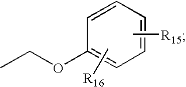
R₁₅ and R₁₆ are selected independently, from the group consisting of hydrogen, alkyl, and halogen;
and A₂ is selected from the group consisting of 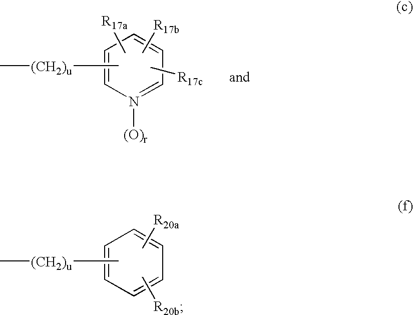
R₁₇ₐ, R_{17b}, and R_{17c} are each independently selected from the group consisting of hydrogen, alkyl, halogen, hydroxy, aryloxy, and hydroxyalkyl;
u is 0, 1, 2, 3, or 4;
r is 0 or 1;
R₂₀ₐ and R_{20b} are independently selected from the group consisting of hydrogen, alkyl, halogen, and aryl; or
R₂₀ₐ and R_{20b} can be taken together with the aryl to which they are attached to form a bicyclic system.

27. A compound according to claim 26, wherein A₂ is

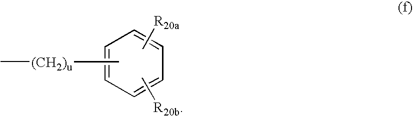

28. A compound according to claim 27, wherein u is 0.

29. A compound according to claim 27, represented by the following formula:

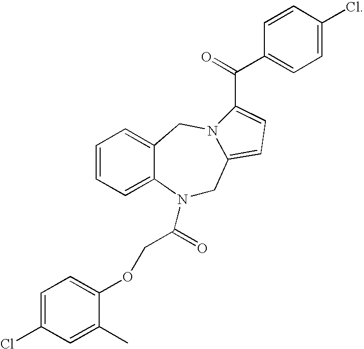

30. A compound according to claim 27, represented by the following formula:

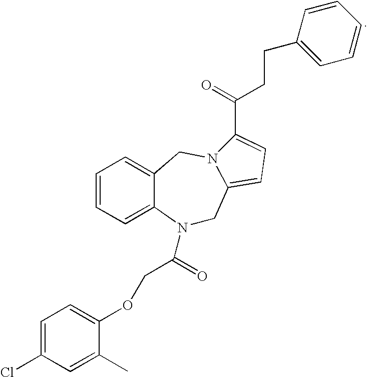

31. A compound according to claim 27, represented by the following formula:

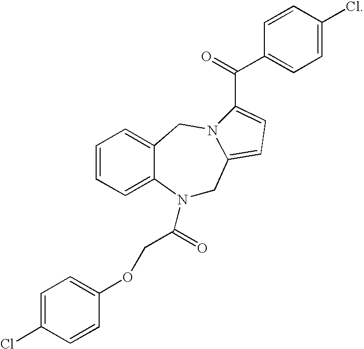

32. A compound according to claim 27, represented by the following formula:

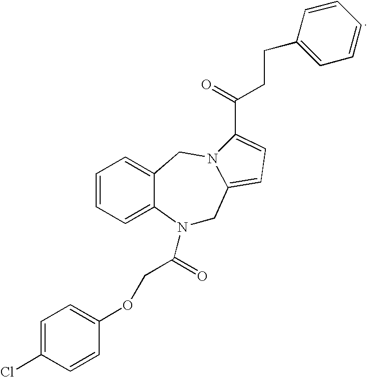

33. A compound according to claim 28, wherein R₂₀ taken together with the aryl to which it is attached form a bicyclic structure.

34. A compound according to claim 33, wherein said bicyclic structure is naphthalene.

35. A compound according to claim 28 represented by the formula:

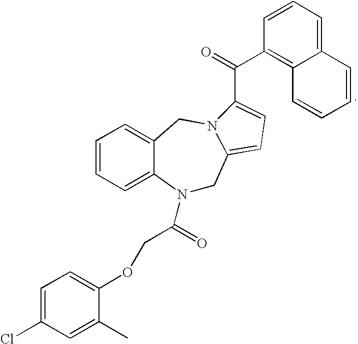

36. A compound according to claim 28, wherein A₂ is

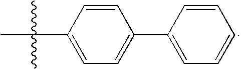

37. A compound according to claim 28, wherein A₂ is

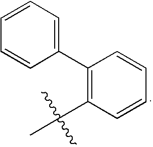

38. A compound according to claim 28 represented by the formula:

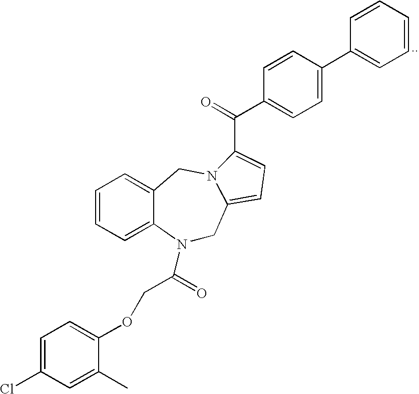

39. A compound according to claim 28 represented by the formula:

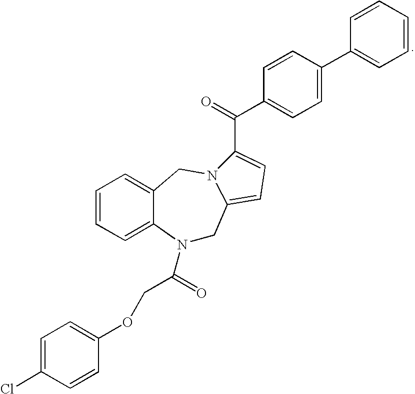

40. A compound according to claim 28 represented by the formula:

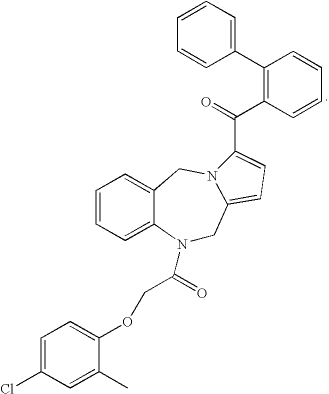

40. A compound according to claim 28 represented by the formula:

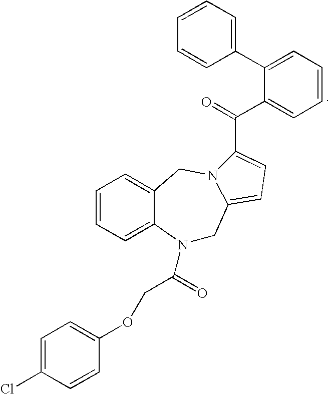

41. A compound according to claim 28 represented by the formula:

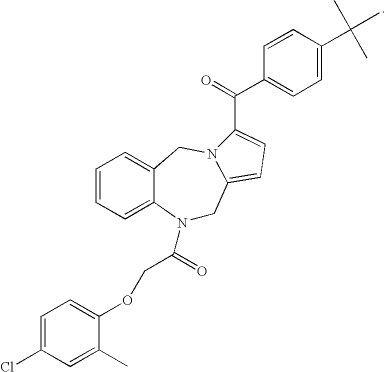

42. A compound according to claim 28 represented by the formula:

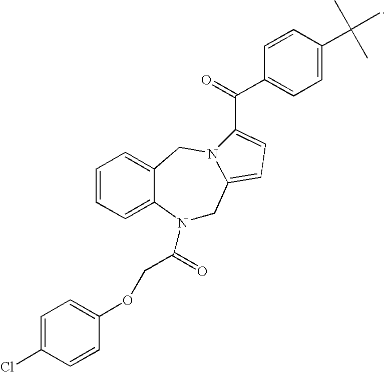

43. A compound according to claim 26, wherein A₂ is

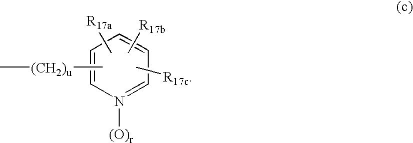

44. A compound according to claim 26, wherein B₂ is 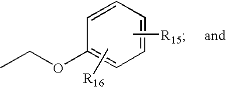
one of R₁₅ or R₁₆ is halogen.

45. A method for preparing a compound of general formula II 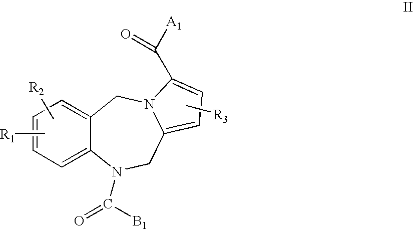
or a pharmaceutically acceptable salt thereof,
wherein
R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];
R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B₁ is selected independently from the group consisting of 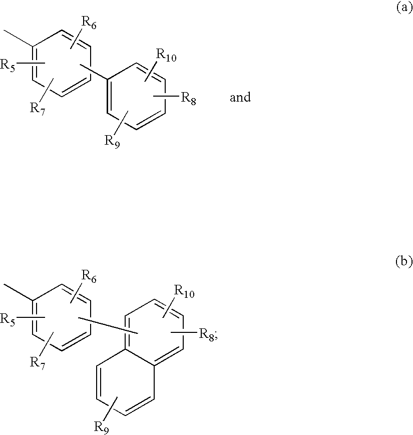
wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxyalkyl, hydroxy(C₁-C₆) alkyl, alkyloxyalkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, (C₁-C₆)alkyl substituted with 1-3 halogen atoms, trihalomethyl, trifluoromethyl, halogen, OCF₃, thioalkyl, thio(C₁-C₆) alkyl, —C(O) alkyl, —C(O)aryl optionally substituted by alkyl; hydroxy, —CH(OH)alkyl, —CH(alkoxy)alkyl, nitro, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCONR₁₃R₁₄, —(CH₂)ₚCOOR₁₂, —CH═NOH, —CH═NO—(C₁-C₆) alkyl, trifluoromethylthio, 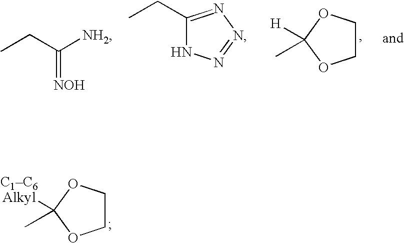
R₁₁ and R₁₂ are independently hydrogen or alkyl;
R₁₃ and R₁₄ are hydrogen or alkyl, or
R₁₃ and R₁₄ can be taken together with the nitrogen to which they are attached to form a 4-6 membered saturated ring optionally containing up to two ring heteroatoms selected from O, S or N;
p is 0 or 1;
A₁ is selected from the group consisting of 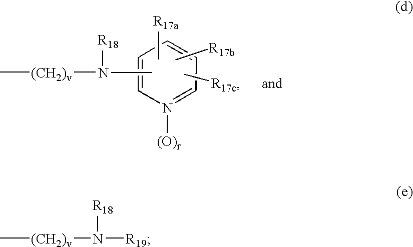
R₁₇ₐ, R_{17b}, and R_{17c} are each independently selected from the group consisting of hydrogen, alkyl, halogen, hydroxy, aryloxy, and hydroxyalkyl;
u is 0, 1, 2, 3, or 4;
v is 1, 2, 3, or 4;
r is 0 or 1;
R₁₈ is hydrogen or alkyl; and
R₁₉ is a cycloalkylamine.
said method comprising:
reacting a compound of formula (2) 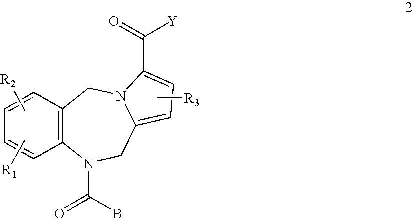
wherein Y is haloalkyl;
with an appropriate amine selected from 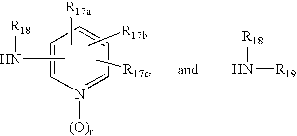
under conditions sufficient to produce the desired compound of formula II.

46. The method of claim 45, wherein the compound of formula (2) is prepared by:
reacting a tricyclic diazepine of formula (1) 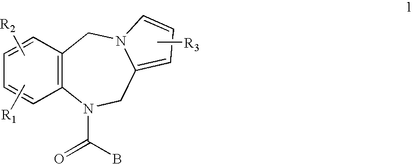
wherein R₁, R₂, and R₃ are defined hereinbefore,
with an acyl halide
XCOY
where X is a halide, and Y is haloalkyl;
under conditions sufficient to produce compound (2).

47. A method of preparing a compound of formula I 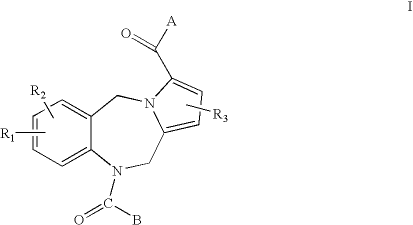
or a pharmaceutically acceptable salt thereof,
wherein
R₁ and R₂ are independently selected from the group consisting of hydrogen, (C₁-C₆) alkyl, halogen, cyano, trifluoromethyl, hydroxyl, (C₁-C₆) alkoxy, -OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];
R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B is B₁ or B₂,
wherein B₁ is selected independently from the group consisting of 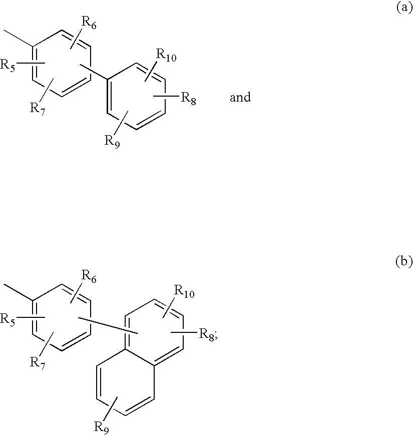
wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxy(C₁-C₆) alkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, halo (C₁-C₆)alkyl including trifluoromethyl, trihalomethyl, halogen, OCF₃, S((C₁-C₆) alkyl), —C(O) alkyl, —C(O)aryl optionally substituted by alkyl; hydroxy, hydroxyalkyl, alkyloxyalkyl, —CH(OH)alkyl, —CH(alkoxy)alkyl, formyl, nitro, thioalkyl, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₁₄, —(CH₂)ₚCONR₁₃R₁₄, —(CH₂)ₚCOOR₁₂, —CH═NOH, —CH═NO—(C₁-C₆) alkyl, trifluoromethylthio, 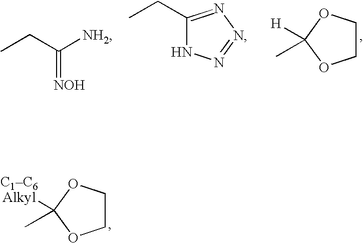
phenyl and naphthyl;
R₁₁ and R₁₂ are each independently hydrogen or alkyl;
R₁₃ and R₁₄ are each independently hydrogen or alkyl,
or R₁₃ and R₁₄ can be taken together with the nitrogen to which they are attached to form a 4-6 membered saturated ring optionally containing up to two atoms selected from O, S or N;
p is 0 or 1;
A is A₁ or A₂, wherein
A₁ is selected from 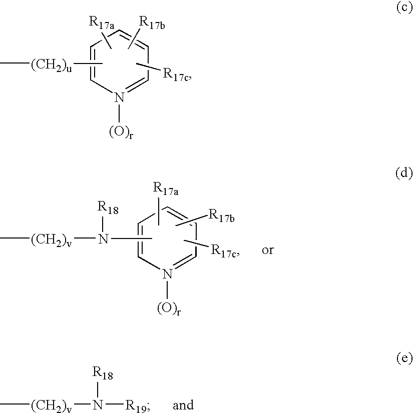
A₂ is selected from 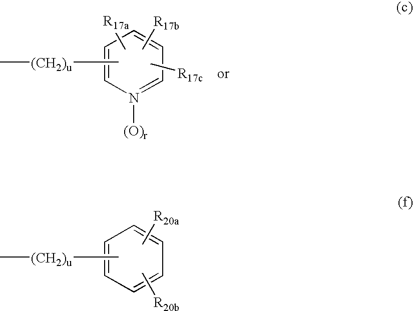
provided that when A is A₂, then B is B₂ wherein B₂ is 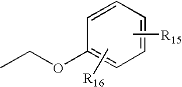
wherein R₁₅ and R₁₆ are selected independently from the group consisting of hydrogen, alkyl, and halogen;
wherein
R₁₇ₐ, R_{17b}, and R_{17c} are each independently selected from the group consisting of hydrogen, alkyl, halogen, hydroxy, aryloxy, and hydroxyalkyl;
u is the integer 0, 1, 2, 3, or 4;
v is the integer 1, 2, 3, or 4;
r is 0 or 1;
R₁₈ is hydrogen or alkyl; and
R₁₉ is a cycloalkylamine.
R₂₀ₐ and R_{20b} are each independently selected from the group consisting of hydrogen, alkyl, halogen, or aryl; or R₂₀ₐ and R_{20b} can be taken together with the aryl to which they are attached to form a bicyclic system; said method comprising:
reacting a tricyclic diazepine of formula (1) 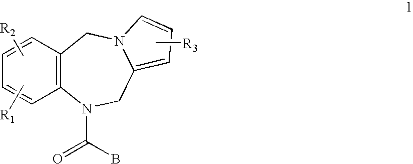
with an acyl halide of formula (4) 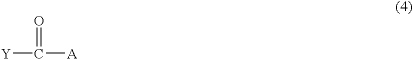
where Y is halogen;
under conditions sufficient to produce the desired compound of formula I.

48. A method of preparing a compound according to formula III 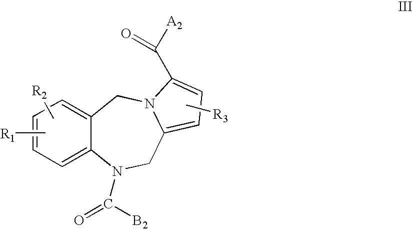
or a pharmaceutically acceptable salt thereof,
wherein
R₁ and R₂ are independently selected from the group consisting of hydrogen, (C₁-C₆) alkyl, halogen, cyano, trifluoromethyl, hydroxyl, (C₁-C₆) alkoxy, -OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];
R₃ is a substituent selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B₂ is 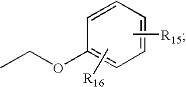
R₁₅ and R₁₆ are selected independently, from the group consisting of hydrogen, alkyl, and halogen;
and A₂ is selected from the group consisting of 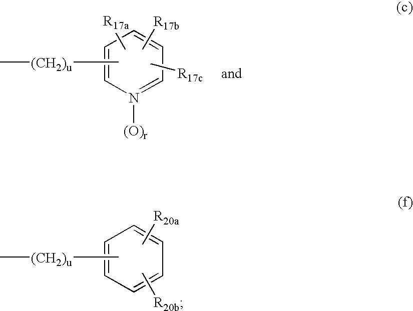
R₁₇ₐ, R_{17b}, and R_{17c} are each independently selected from the group consisting of hydrogen, alkyl, halogen, hydroxy, aryloxy, and hydroxyalkyl;
u is 0, 1, 2, 3, or 4;
r is 0 or 1;
R₂₀ₐ and R_{20b} are independently selected from the group consisting of hydrogen, alkyl, halogen, and aryl; or
R₂₀ₐ and R_{20b} can be taken together with the aryl to which they are attached to form a bicyclic system;
said method comprising:
reacting a tricyclkic diazepine of formula (5) 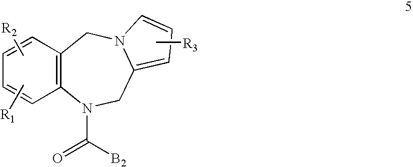
with an acid halide of formula 6
A₂COY (6)
wherein Y is halogen;
under conditions to produce a compound according to formula III.

49. A method for making a compound of formula (I) 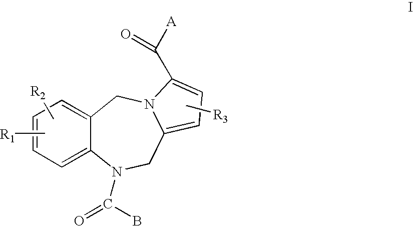
or a pharmaceutically acceptable salt thereof,
wherein
R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];
R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B is B₁ or B₂,
wherein B₁ is selected independently from the group consisting of 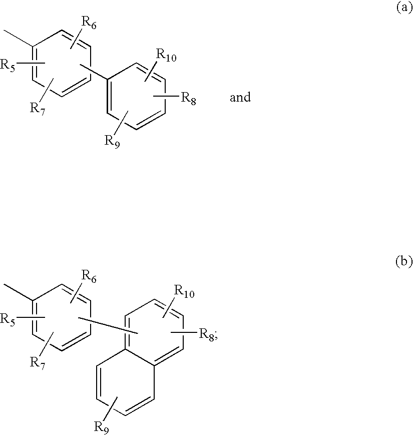
wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxy(C₁-C₆) alkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, halo (C₁-C₆)alkyl including trifluoromethyl, trihalomethyl, halogen, OCF₃, S((C₁-C₆) alkyl), —C(O) alkyl, —C(O)aryl optionally substituted by alkyl; hydroxy, hydroxyalkyl, alkyloxyalkyl, —CH(OH)alkyl, —CH(alkoxy)alkyl, formyl, nitro, thioalkyl, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚCN, (C₁-C₆) alkylamino, di-(C₁-C₆) alkylamino, (C₁-C₆) alkyl di-(C₁-C₆) alkylamino, —(CH₂)ₚNR₁₃R₄, —(CH₂)ₚCONR₁₃R₁₄, —(CH₂)ₚCOOR₁₂, —CH═NOH, —CH═NO—(C₁-C₆) alkyl, trifluoromethylthio, 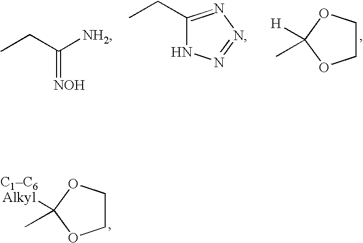
phenyl and naphthyl;
R₁₁ and R₁₂ are each independently hydrogen or alkyl;
R₁₃ and R₁₄ are each independently hydrogen or alkyl,
or R₁₃ and R₁₄ can be taken together with the nitrogen to which they are attached to form a 4-6 membered saturated ring optionally containing up to two atoms selected from O, S or N;
p is 0 or 1;
A is A₁ or A₂, wherein
A₁ is selected from 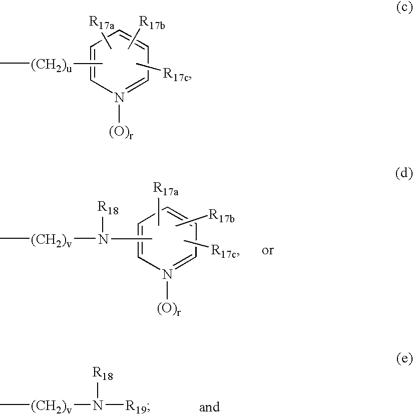
A₂ is selected from 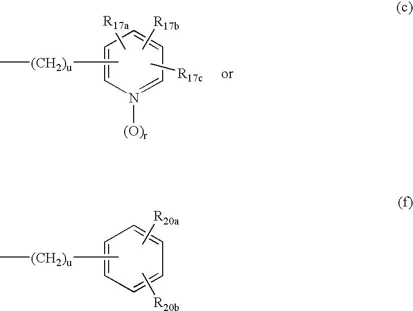
provided that when A is A₂, then B is B₂ wherein B₂ is 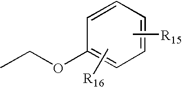
wherein R₁₅ and R₁₆ are selected independently from the group consisting of hydrogen, alkyl, and halogen;
wherein
R₁₇ₐ, R_{17b}, and R_{17c} are each independently selected from the group consisting of hydrogen, alkyl, halogen, hydroxy, aryloxy, and hydroxyalkyl;
u is the integer 0, 1, 2, 3, or 4;
v is the integer 1, 2, 3, or 4;
r is 0 or 1;
R₁₈ is hydrogen or alkyl; and
R₁₉ is a cycloalkylamine.
R₂₀ₐ and R_{20b} are each independently selected from the group consisting of hydrogen, alkyl, halogen, or aryl; or R₂₀ₐ and R_{20b} can be taken together with the aryl to which they are attached to form an aromatic bicycle having up to 10 total ring atoms;
said method comprising
subsequent reaction of the intermediate of formula (26) 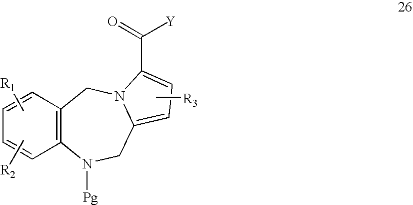
where Y is Cl, with an appropriate amine selected from 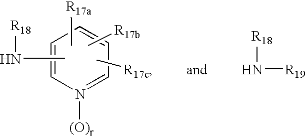
under the conditions sufficient to provide the intermediate of formula (27)

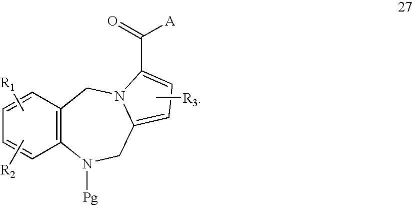

50. The method of claim 104, further comprising deprotecting the compound of formula (27) to yield the intermediate of formula (28) 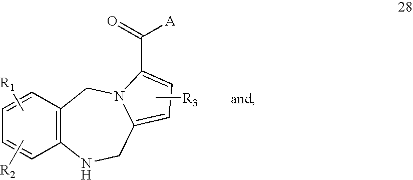
then acylating the intermediate of formula (28) to the desired product of formula (I).

51. The method of claim 50 wherein said compound of formula (26) is prepared by reacting a tricyclic diazepine of formula (25) 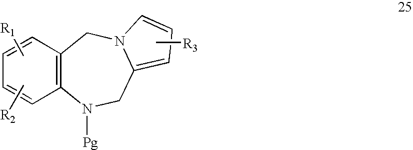
wherein
R₁, R₂ and R₃ are defined hereinbefore,
Pg is a protecting group;
with a an acid chloride under conditions sufficient to provide the desired intermediate of formula (26).

52. A method for making a compound of formula (I) 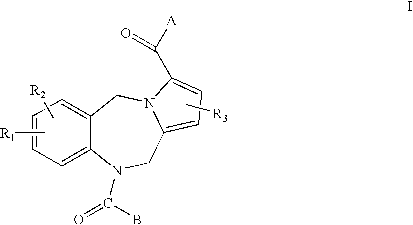
or a pharmaceutically acceptable salt thereof,
wherein
R₁ and R₂ are independently selected from the group consisting of hydrogen, (C₁-C₆) alkyl, halogen, cyano, trifluoromethyl, hydroxyl, (C₁-C₆) alkoxy, —OCF₃, carboxy, (C₁-C₆ alkoxy)carbonyl, —CONH₂, —CONH[(C₁-C₆) alkyl], —CON[(C₁-C₆) alkyl]₂, amino, (C₁-C₆) alkylamino, and —NHCO[(C₁-C₆) alkyl];
R₃ is selected from the group consisting of hydrogen, (C₁-C₆) alkyl, (C₁-C₆) alkoxy, hydroxy, amino, (C₁-C₆) alkylamino, —C(O)(C₁-C₆)alkyl, and halogen;
B is B₁ or B₂,
wherein B₁ is selected independently from the group consisting of 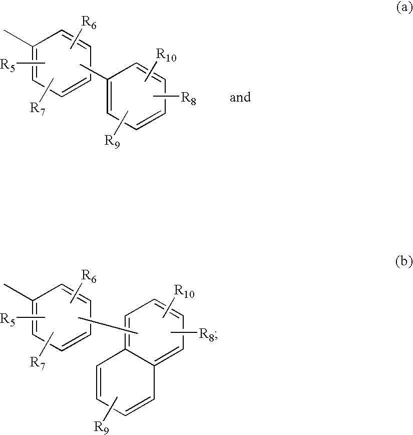
wherein R₅, R₆, R₇, R₈, R₉ and R₁₀ are independently, selected from the group consisting of hydrogen, alkyl, (C₁-C₆)alkyl, alkoxy, (C₁-C₆) alkoxy, hydroxy(C₁-C₆) alkyl, (C₁-C₆)alkoxy(C₁-C₆)alkyl, (C₂-C₇) acyloxy (C₁-C₆)alkyl, (C₁-C₆alkyl) carbonyl, (C₂-C₆) alkenyl, (C₂-C₆) alkynyl, (C₃-C₈) cycloalkyl, formyl, (C₃-C₈)cycloalkylcarbonyl, carboxy, (C₁-C₆)alkoxycarbonyl, (C₃-C₈cycloalkyl) oxycarbonyl, aryl(C₁-C₆)alkyloxycarbonyl, carbamoyl, —O—CH₂—CH═CH₂, halo (C₁-C₆)alkyl including trifluoromethyl, trihalomethyl, halogen, OCF₃, S((C₁-C₆) alkyl), —C(O) alkyl, —C(O)aryl optionally substituted by alkyl; hydroxy, hydroxyalkyl, alkyloxyalkyl, —CH(OH)alkyl, —CH(alkoxy)alkyl, formyl, nitro, thioalkyl, —SO₂alkyl, (C₁-C₆) alkylsulfonyl, aminosulfonyl, (C₁-C₆) alkylaminosulfonyl, —SO₂NHR₁₁, —SO₂N(R₁₁)₂, —OC(O)N[(C₁-C₆)alkyl]₂, —CONH[(C₁-C₆) alkyl], —CON[(C₁-C₆) alkyl]₂, —(CH₂)ₚNR₁₃R₁₄, —di-(C₁-C₆) alkylamino, (C₁-C₆) alkyl di-(C₁-C₆) alkylamino, —(CH₂)ₚNR₁₃R₁₄, —(CH₂)ₚCONR₁₃R₁₄, —(CH₂)ₚCOOR₁₂, —CH═NOH, —CH═NO—(C₁-C₆) alkyl, trifluoromethylthio, 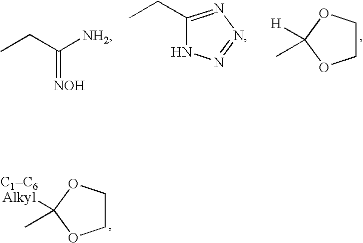
phenyl and naphthyl;
R₁₁ and R₁₂ are each independently hydrogen or alkyl;
R₁₃ and R₁₄ are each independently hydrogen or alkyl,
or R₁₃ and R₁₄ can be taken together with the nitrogen to which they are attached to form a 4-6 membered saturated ring optionally containing up to two atoms selected from O, S or N;
p is 0 or 1;
A is A₁ or A₂, wherein
A₁ is selected from 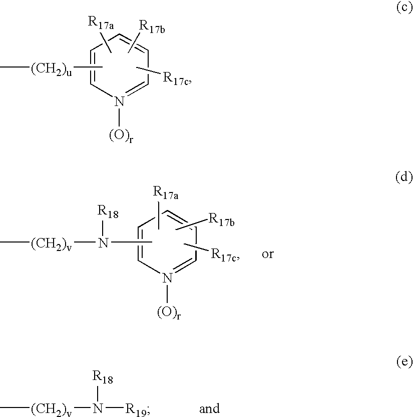
A₂ is selected from 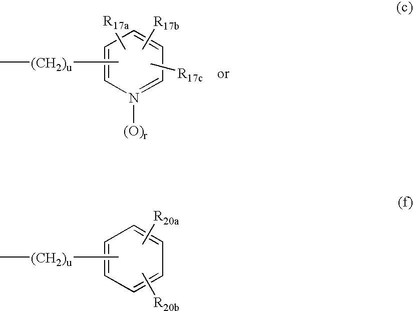
provided that when A is A₂, then B is B₂ wherein B₂ is 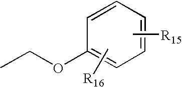
wherein R₁₅ and R₁₆ are selected independently from the group consisting of hydrogen, alkyl, and halogen;
wherein
R₁₇ₐ, R_{17b}, and R_{17c} are each independently selected from the group consisting of hydrogen, alkyl, halogen, hydroxy, aryloxy, and hydroxyalkyl;
u is the integer 0, 1, 2, 3, or 4;
v is the integer 1, 2, 3, or 4;
r is 0 or 1;
R₁₈ is hydrogen or alkyl; and
R₁₉ is a cycloalkylamine.
R₂₀ₐ and R_{20b} are each independently selected from the group consisting of hydrogen, alkyl, halogen, or aryl; or R₂₀ₐ and R_{20b} can be taken together with the aryl to which they are attached to form an aromatic bicycle having up to 10 total ring atoms;
said method comprising
treating a compound of formula (25) with an acid chloride of formula (4)
ACOY 4
under the conditions sufficient to yield the amide of formula (27) 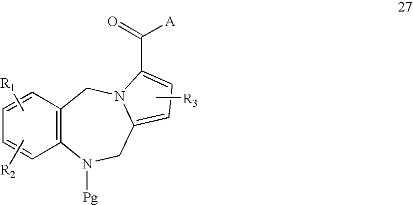
wherein A is A₂ as defined hereinbefore.

53. The method of claim 52, further comprising:
deprotecting the compound of formula (27) to yield the intermediate of formula (28) 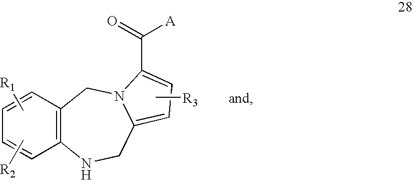
then acylating the intermediate of formula (28) to the desired product of formula (I).
